(19) United States
(12) Patent Application Publication
(10) Pub. No.: US 2026/0125411 A1
(43) Pub. Date: May 7, 2026

(54) COMPOUND EMITTING LIGHT UNDER SPECIFIC CONDITIONS, AND METHOD FOR DETECTING CANCER STEM CELLS USING SAME

(71) Applicants: Kyoto University, Kyoto (JP); National University Corporation Tokai National Higher Education And Research System, Aichi (JP)

(72) Inventors: Koji Miki, Kyoto (JP); Masahiro Oe, Kyoto (JP); Kanae Suzuki, Kyoto (JP); Huiying Mu, Kyoto (JP); Yoshifumi Ueda, Kyoto (JP); Yasuo Mori, Kyoto (JP); Hiroshi Yukawa, Aichi (JP); Kouichi Ohe, Kyoto (JP); Koki Miki, Kyoto (JP); Yoshinobu Baba, Aichi (JP)

(73) Assignees: Kyoto University, Kyoto (JP); National University Corporation Tokai National Higher Education And Research System, Aichi (JP)

(21) Appl. No.: 19/144,262

(22) PCT Filed: Dec. 27, 2023
(86) PCT No.: PCT/JP2023/047011

§ 371 Date: Jun. 27, 2025

(30) Foreign Application Priority Data
Dec. 28, 2022 (JP) 2022-211614

(51) Int. Cl. C07H 17/04; A61K 49/00; A61P 35/00; G01N 33/50

(52) U.S. Cl. CPC C07H 17/04; A61K 49/0052; A61P 35/00; G01N 33/5005

(57) ABSTRACT

The present invention provides a compound with which a molecular probe capable of distinguishing NSCs from CSCs can be produced, and a method for detecting cancer stem cells using the compound. The present invention relates to a compound having a formyl group, a substrate moiety, a quenching moiety, and a luminescent moiety that is non-luminous due to the quenching moiety, wherein the luminescent moiety may be rendered luminous by detaching the substrate moiety by a substrate-degrading enzyme and also converting the formyl group to a carboxy group by an aldehyde dehydrogenase to allow the quenching moiety to dissociate.

### TECHNICAL FIELD

The present invention relates to a compound that is rendered luminous under specific conditions and a method for detecting cancer stem cells using the compound.

### BACKGROUND ART

Cancer cells are known to contain approximately a few percent of cancer stem cells (CSCs) which exhibit resistance to anticancer drugs and contribute to cancer metastasis. Visualizing CSCs may be useful in developing truly effective anticancer drugs and diagnosing highly malignant cancer tissue. Aldehyde dehydrogenase 1A1 (ALDH1A1) is known as a biomarker that is highly expressed in cells with stemness properties. Molecular probes capable of detecting ALDH1A1 activity may be useful for identifying CSCs, but few examples have been reported.

In 1999, the ALDH1A1-responsive molecular probe ALDEFLUOR™, which was later made commercially available, was announced (Non-Patent Literature 1), making it possible to identify CSCs. Very recently, a wavelength-shifting probe (Non-Patent Literature 2) and turn-on probes (Non-Patent Literatures 3 to 5) have been reported, each of which compensates for the shortcomings of ALDEFLUOR™, which is an always-on probe. Thus, several molecular probes capable of detecting ALDH1A1 (or ALDH1) activity have been developed to date. However, ALDH1A1, which is a biomarker for stem cells, is also expressed in normal stem cells (NSCs) contained in normal tissue. In other words, the molecular probes developed so far cannot distinguish NSCs from CSCs as illustrated in FIG. 10.

Therefore, in clinical settings, the molecular probes developed so far have the disadvantage of not being able to eliminate false positives caused by NSCs when staining tissue sections containing normal tissue and/or blood cells to determine whether they contain highly malignant tumors containing a large number of CSCs.

### CITATION LIST

### Non-Patent Literature

Non-Patent Literature 1: R. W. Storms, et al. Proc. Natl. Acad. Sci. USA 1999, 96, 9118
Non-Patent Literature 2: S. Maity, et al. Chem. Sci. 2017, 8, 7143. DOI: 10.1039/c7sc03017g
Non-Patent Literature 3: C. Anorma, et al. ACS Cent. Sci. 2018, 4, 1045. DOI: 10.1021/acscentsci.8b00313; T. E. Bearrood, et al. Bioconjugate Chem. 2020, 31, 224. DOI: 10.1021/acs.bioconjchem.9b00723
Non-Patent Literature 4: M. Oe, et al. ACS Sens. 2021, 6, 3320. DOI: 10.1021/acssensors.1c01136
Non-Patent Literature 5: Q. W. Wang, et al. Anal. Chem. 2022, 94, 49, 17328-17333

### SUMMARY OF INVENTION

### Technical Problem

The present invention aims to provide a compound with which a molecular probe capable of distinguishing NSCs from CSCs can be produced, and a method for detecting cancer stem cells using the compound.

### Solution to Problem

The present inventors conducted research into compounds capable of detecting ALDH1A1 (or ALDH1) activity, and focused on β-galactosidase (β-gal), which is known to be expressed in cancer cells but not in normal cells. The inventors then found that such a sugar chain may be introduced into a compound containing a formyl group and a quenching moiety in order to distinguish NSCs from CSCs, thus completing the present invention, as illustrated in FIG. 11.

Specifically, Embodiment 1 of the present invention relates to a compound, having a formyl group, a substrate moiety, a quenching moiety, and a luminescent moiety that is non-luminous due to the quenching moiety, wherein the luminescent moiety may be rendered luminous by detaching the substrate moiety by a substrate-degrading enzyme and also converting the formyl group to a carboxy group by an aldehyde dehydrogenase to allow the quenching moiety to dissociate.

Embodiment 2 of the present invention (2) relates to the compound according to Embodiment 1 of the present invention, wherein the substrate moiety is a sugar chain, an amino acid, a peptide chain, a boryl group, a phosphoryl group, an aryl methyl group, or an acyl group.

Embodiment 3 of the present invention relates to the compound according to Embodiment 2 of the present invention, wherein the sugar chain is D-galactose, and the sugar chain-degrading enzyme is β-galactosidase.

Embodiment 4 of the present invention relates to the compound according to any one of Embodiments 1 to 3 of the present invention, wherein the luminescent moiety is able to emit near-infrared light.

Embodiment 5 of the present invention relates to a compound represented by the following formula:

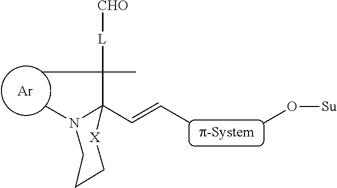

wherein Ar is an aromatic ring, X is an oxygen atom, a nitrogen atom, a sulfur atom, or a selenium atom, L is a linker, Su is a substrate moiety, and the π-system is a π-conjugated system.

Embodiment 6 of the present invention relates to the compound according to Embodiment 5 of the present invention, wherein the aromatic ring is represented by the following formula:

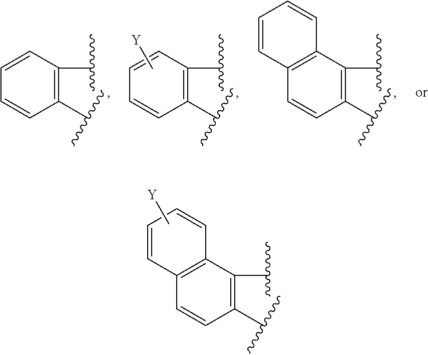

wherein Y is a halogen group, SO₃Na, SO₃H, or COOR.

Embodiment 7 of the present invention relates to the compound according to Embodiment 5 or 6 of the present invention, wherein the substrate moiety is a sugar chain, an amino acid, a peptide chain, a boryl group, a phosphoryl group, an aryl methyl group, or an acyl group.

Embodiment 8 of the present invention relates to the compound according to any one of Embodiments 5 to 7 of the present invention, wherein the π-conjugated system is represented by the following formula:

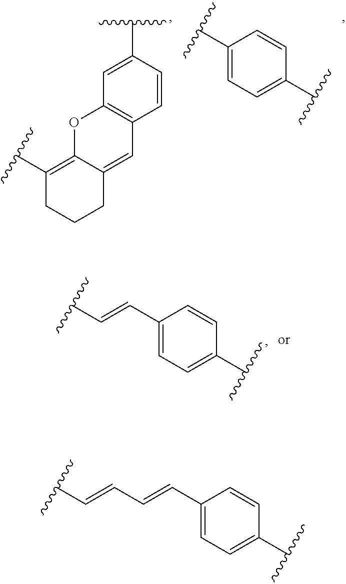 [SMILES: *C1=C2Oc3cc(*)ccc3C=C2CCC1 ; *c1ccc(*)cc1 ; */C=C/c1ccc(*)cc1 ; */C=C/C=C/c1ccc(*)cc1]

wherein the aromatic ring optionally has an electron-withdrawing group.

Embodiment 9 of the present invention relates to the compound according to any one of Embodiments 5 to 8 of the present invention, wherein the compound is represented by the following formula:

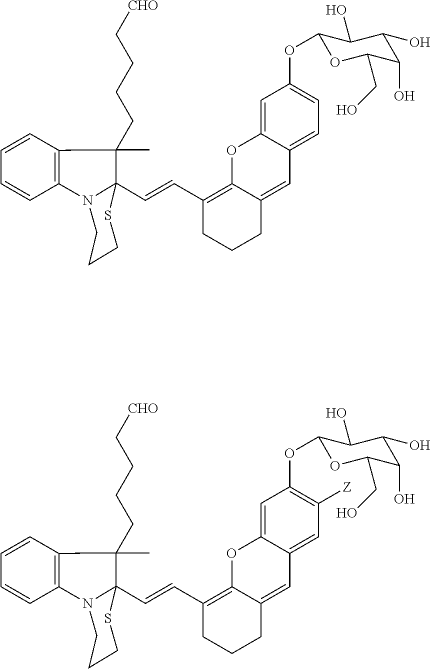

wherein Z is an electron-withdrawing group.

Embodiment 10 of the present invention relates to a cancer stem cell detection reagent, containing the compound according to any one of Embodiments 1 to 9 of the present invention.

Embodiment 11 of the present invention relates to a method for detecting cancer stem cells, the method including: administering the cancer stem cell detection reagent according to Embodiment 10 of the present invention to cells; and irradiating the cells with near-infrared light to detect cancer stem cells by emission from the compound.

Embodiment 12 of the present invention relates to the method for detecting cancer stem cells according to Embodiment 11 of the present invention, wherein the cells are contained in biological tissue and are detected in vivo or in vitro.

### Advantageous Effects of Invention

The compound according to the present invention can be used to produce a molecular probe capable of distinguishing NSCs from CSCs, thus making it possible to detect cancer stem cells.

### BRIEF DESCRIPTION OF DRAWINGS

FIG. 1-1 shows changes in fluorescence intensity when CHO_βgal (20 μM, phosphate buffer solution (pH=7.4)) synthesized in Example 1 was treated with (a) no enzyme (control), (b) ALDH1A1 alone, (c) β-galactosidase alone, and (d) ALDH1A1 and β-galactosidase; and (e) changes over time in fluorescence intensity at 710 nm.

FIG. 1-2 shows changes in fluorescence intensity of (a) the compound synthesized in Synthesis Example 9 and (b) COOH_OH produced by the enzymatic response of CHO_βgal.

FIG. 2 shows the responsiveness (37° C., 60 min) of CHO_βgal synthesized in Example 1 to biochemical substances and oxidizing agents.

FIG. 3 shows the results of a cytotoxicity test of CHO_βgal synthesized in Example 1 to (a) SUIT-2, (b) KATO-III, (c) HUVEC, and (d) NIH-3T3.

FIG. 4 shows confocal laser microscope images of (a) SUIT-2, (b) KATO-III, (c) NIH-3T3, and (d) HUVEC, each treated with C5S-A (left) and CHO_βgal (right).

FIG. 5 shows confocal laser microscope images of (a) SUIT-2, (b) KATO-III, (c) A549, and (d) OVK18, each co-stained with CHO_βgal synthesized in Example 1 and Aldefluor™ (left) or with CHO_βgal and FDG (right).

FIG. 6 shows fluorescence microscope images of (a) tumor tissue stained with CHO_βgal and (b) tumor tissue treated with the inhibitor disulfiram and then stained with CHO_βgal.

FIG. 7 shows fluorescence microscope images or optical microscope images of the boundary between tumor tissue and normal cells stained with (c) CHO_βgal, (d) C5S-A, or (e) hematoxylin-eosin.

FIG. 8 shows the results (fluorescence images) of an excised lung of a lung metastasis model mouse treated with C5S-A and CHO_βgal.

FIG. 9 shows the results (fluorescence images) of an excised lung of a lung metastasis model mouse treated with C5S-A and CHO_βgal and the plots of fluorescence intensity.

FIG. 10 illustrates a conventional model using an ALDH1-responsive molecular probe.

FIG. 11 illustrates a model of one or more embodiments of the present invention.

### DESCRIPTION OF EMBODIMENTS

A compound according to a first embodiment of the present invention is characterized by having a formyl group, a substrate moiety, a quenching moiety, and a luminescent moiety that is non-luminous due to the quenching moiety, wherein the luminescent moiety may be rendered luminous by detaching the substrate moiety by a substrate-degrading enzyme and also converting the formyl group to a carboxy group by an aldehyde dehydrogenase to allow the quenching moiety to dissociate.

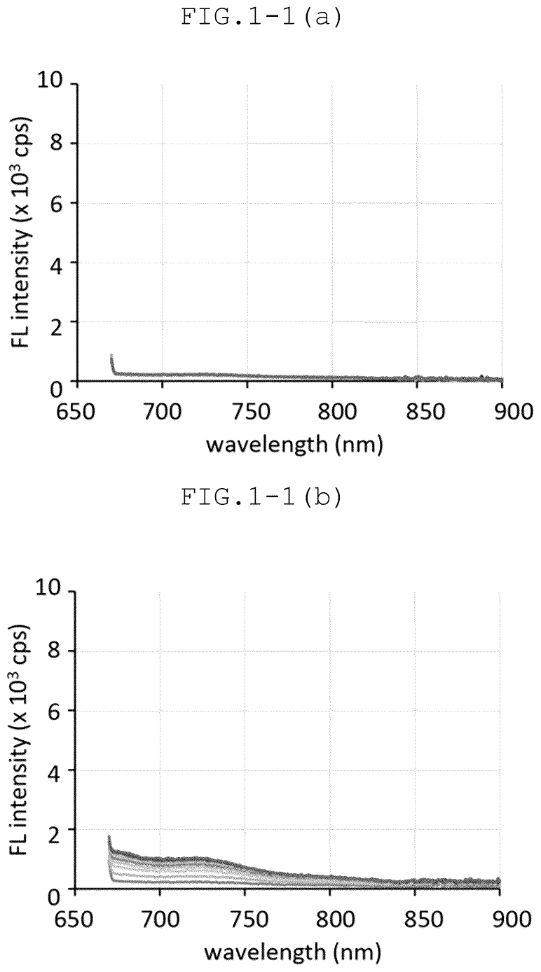

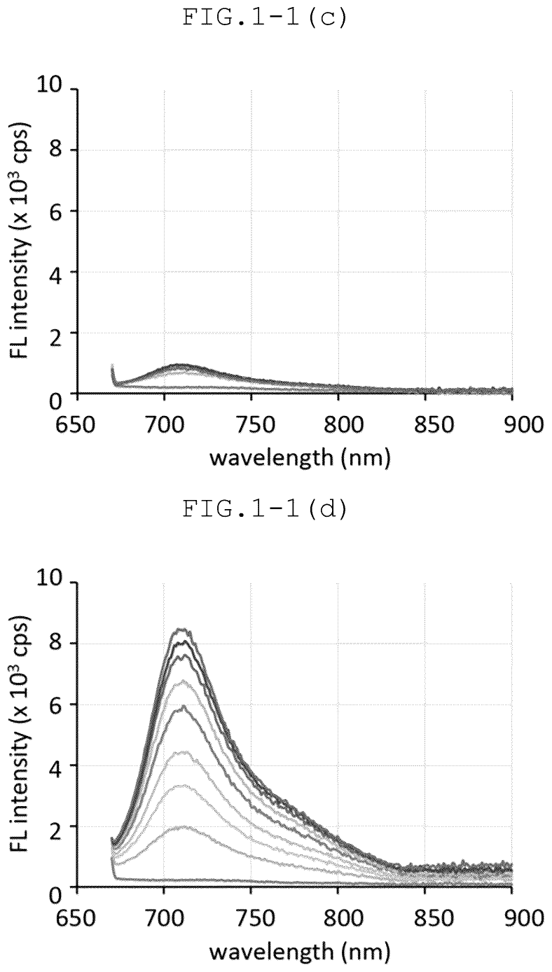

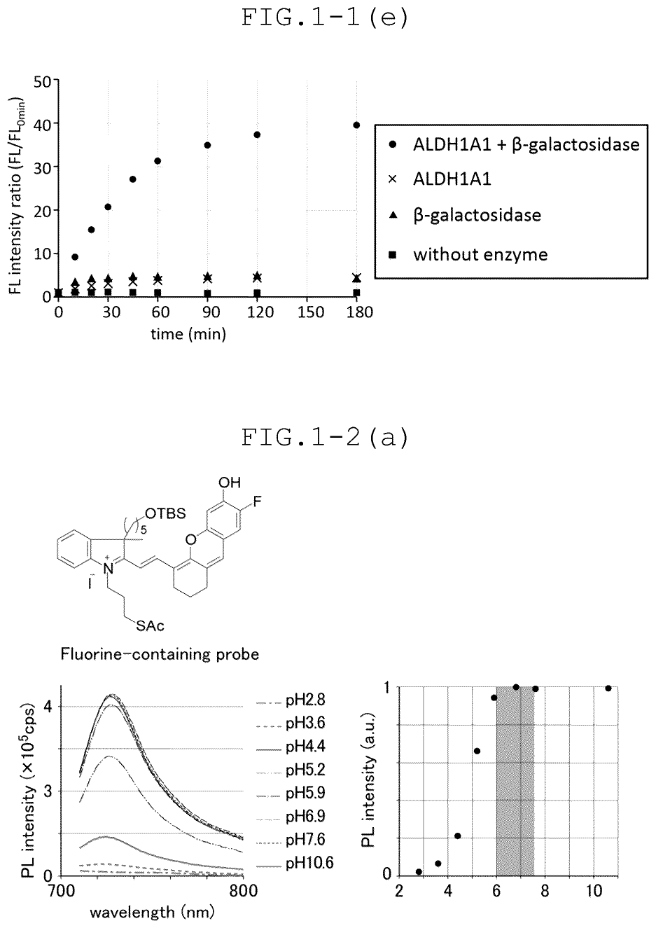

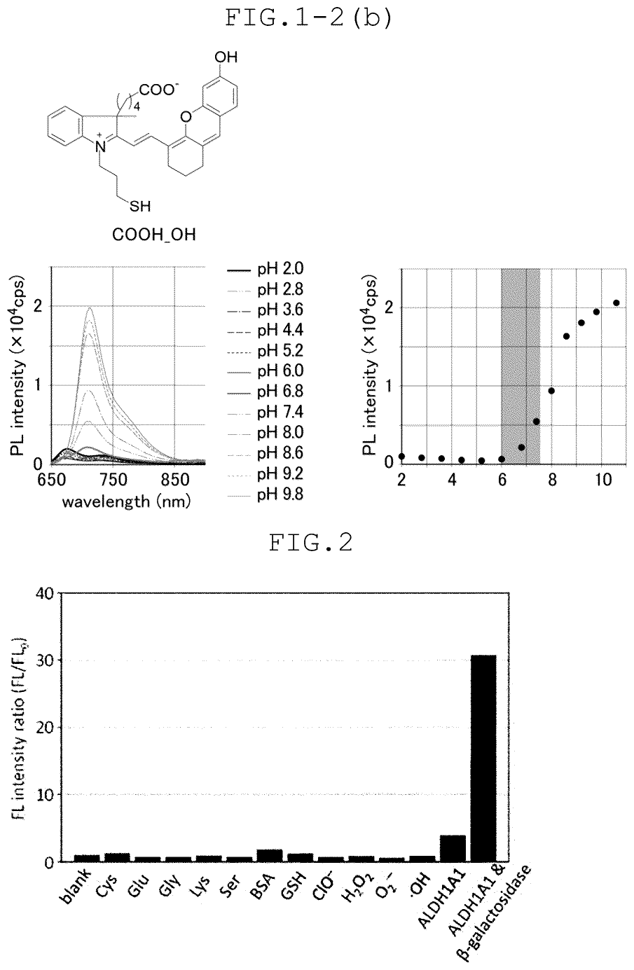

The luminescence mechanism of the compound of the present invention is described based on CHO_βgal, which is an example of a compound of the present invention in which the substrate moiety is D-galactose, the quenching moiety is S, and the luminescent moiety is a structure including indole and a xanthene skeleton linked via a vinylene group. The D-galactose in CHO_βgal can be detached by β-galactosidase in cancer stem cells to produce CHO_OH. The formyl group can be converted to a carboxy group by the aldehyde dehydrogenase ALDH1A1, a biomarker that is highly expressed in cells with stemness properties. The conversion to a carboxy group is accompanied by dissociation of the quenching moiety S (dissociation of the bond between S and C) to form an iminium, which is then conjugated with the xanthene skeleton via the vinylene to change the luminescent moiety from non-luminous to luminous. This compound emits fluorescence when irradiated with near-infrared light. According to such a mechanism, only cancer stem cells, which correspond to both cancer cells containing β-galactosidase and stem cells containing ALDH1A1, can be detected. It should be noted that without detachment of D-galactose from CHO_βgal, little emission occurs when the formyl group is converted to a carboxy group by the aldehyde dehydrogenase ALDH1A1, followed by dissociation of the quenching moiety S to form an iminium, which is then conjugated with the xanthene skeleton via the vinylene (see FIG. 1-1(b)).

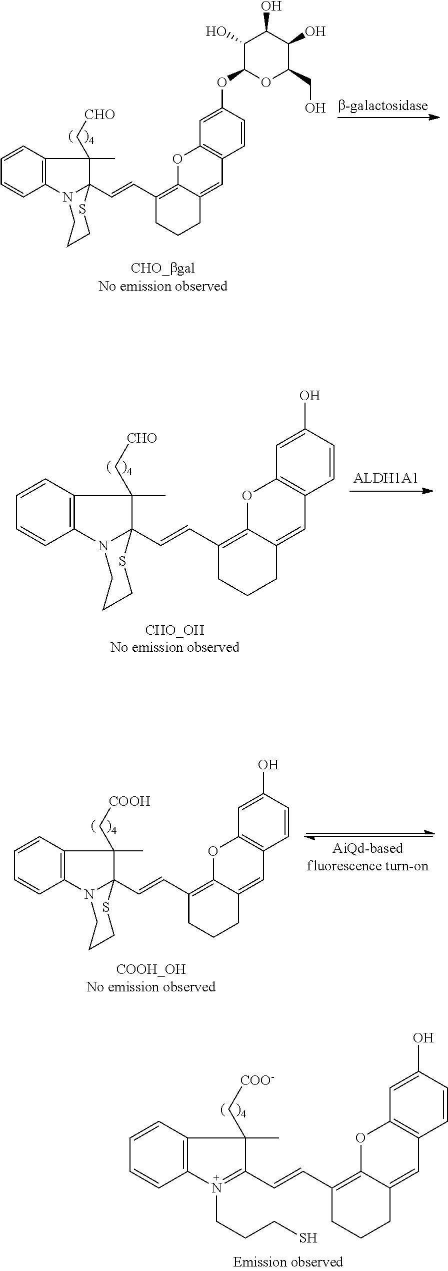

### <Aldehyde Dehydrogenase>

The aldehyde dehydrogenase may be any enzyme that can convert aldehyde to a carboxy group. Examples include ALDH1A1, ALDH1A2, ALDH1A3, ALDH2, ALDH3A1, and ALDH7A1. Of these, ALDH1A1 is preferred.

### <Formyl Group>

The formyl group can be oxidized to a carboxy group by an aldehyde dehydrogenase in cancer stem cells, reducing the ability of the compound to cross the cell membrane from the cancer stem cells to the outside of the cells. The formyl group is preferably bound to the aromatic ring of the luminescent moiety via a linker. Examples of the linker include C1-C10, preferably C2-C6, alkylene chains, and arylenes. An alkylene chain may contain an ether bond, an ester bond, an amide bond, or other bonds.

### <Substrate Moiety and Substrate-Degrading Enzyme>

The substrate moiety can be detached by a substrate-degrading enzyme, e.g., to form a hydroxy group in the π-conjugated system in the luminescent moiety. The formation of a hydroxy group serves to drastically improve the luminescence of the luminophore. Examples of the substrate moiety include sugar chains, amino acids, peptide chains, boryl groups, phosphoryl groups, aryl methyl groups, and acyl groups. Among these, sugar chains or amino acids are preferred from the perspective of targeting enzymes overexpressed in cancer cells. The substrate moiety is preferably bound to the luminescent moiety via O, N, or the like which can be degraded by the substrate-degrading enzyme.

Examples of the sugar chains include D-galactose, D-glucuronic acid, and N-acetylglucosamine. Of these, D-galactose is preferred in that β-galactosidase is overexpressed in cancer cells.

The substrate-degrading enzyme functions to detach the substrate moiety from the compound of the present invention. The substrate-degrading enzyme may be any enzyme present in cancer cells. Examples include β-galactosidase when the substrate moiety is D-galactose, β-glucuronidase when the substrate moiety is D-glucuronic acid, and N-acetyl-β-glucosaminidase when the substrate moiety is N-acetylglucosamine.

### <Quenching Moiety and Luminescent Moiety>

The quenching moiety functions to render the luminescent moiety non-luminous. In CHO_βgal, the —SH group has been bound to the carbon-nitrogen double bond to form a single bond and block the conjugated system, thereby changing it from luminous to non-luminous. Examples of the quenching moiety include, in addition to sulfur, oxygen, selenium, and nitrogen.

The luminescent moiety may be any moiety that can transition from a non-luminous moiety having a chemical structure with a blocked π-conjugated system to a luminous moiety having a chemical structure with a continuous π-conjugated system. The combination of an aromatic heterocycle, an aryl iminium, and vinylene linking them in the compound at the right end of the reaction scheme constitutes the luminescent moiety. The quenching moiety dissociates due to the carboxy group produced from the formyl group to form an iminium containing the nitrogen atom of the five-membered ring. Then, the aromatic ring, iminium, and π-conjugated system are conjugated through one to two vinylene groups to form an extended π-conjugated system, which emits light. In view of application to biological imaging, the luminescent moiety preferably absorbs and emits light with a wavelength of 650 to 900 nm, and preferably emits near-infrared light.

In CHO_βgal, examples of the heterocycle corresponding to indole on the left side include benzoindole in addition to indole. Examples of the aromatic ring constituting the heterocycle include benzene, naphthalene, anthracene, and thiophene rings. Meanwhile, examples of the π-conjugated skeleton corresponding to a xanthene skeleton on the right side include aryl and aryl vinylene groups in addition to xanthene skeletons. Among these, xanthene skeleton, aryl vinylene, and other groups are preferred in that they emit light when irradiated with near-infrared light. It should be noted that these aromatic rings may have a substituent such as a halogen group, SO₃Na, SO₃H, or COOR.

In particular, the compound of the present invention is preferably a compound having a substrate moiety; a luminescent moiety including a π-conjugated system bound to the substrate moiety via an oxy group; a quenching moiety forming an additional fused ring structure on a nitrogen atom-containing heterocycle fused to an aromatic ring; and a formyl group bound via an alkylene to the nitrogen atom-containing heterocycle fused to the aromatic ring, wherein the π-conjugated system is linked to the nitrogen atom-containing heterocycle via one to three vinylene groups.

A compound according to a second embodiment of the present invention is characterized by being represented by the following formula:

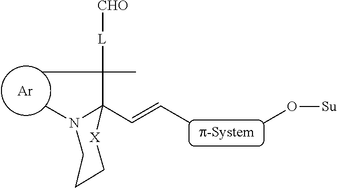

wherein Ar is an aromatic ring, X is an oxygen atom, a nitrogen atom, a sulfur atom, or a selenium atom, L is a linker, Su is a substrate moiety, and the I-system is a π-conjugated system.

In the chemical formula, Ar is an aromatic ring. The aromatic ring may be, for example,

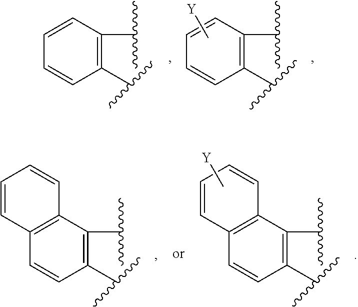

Here, Y is a halogen group, SO₃Na, SO₃H, or COOR.

In the chemical formula, Su is a substrate moiety. Examples of the substrate moiety include sugar chains, amino acids, peptide chains, boryl groups, phosphoryl groups, aryl methyl groups, and acyl groups. Among these, sugar chains or amino acids are preferred from the perspective of targeting enzymes overexpressed in cancer cells.

Examples of the sugar chains include D-galactose, D-glucuronic acid, and N-acetylglucosamine. Of these, D-galactose is preferred in that β-galactosidase is overexpressed in cancer cells.

In the chemical formula, L is a linker. Examples of the linker include C1-C10 alkylene groups, and arylenes. The number of carbon atoms in the alkylene group used is preferably 2 to 6. The alkylene chain may contain an ether bond, an ester bond, an amide bond, or other bonds.

In the chemical formula, the π-system is a π-conjugated system. The π-conjugated system may be, for example,

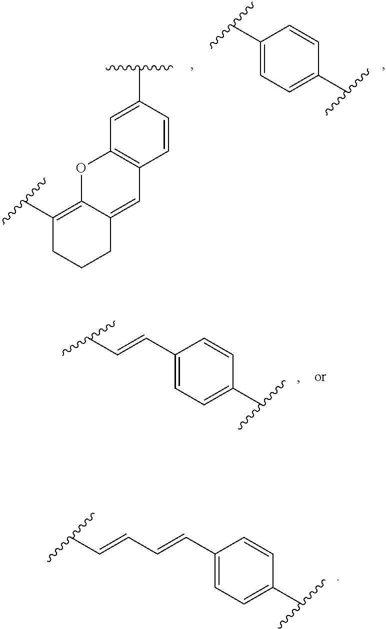 [SMILES: *C1=C2Oc3cc(*)ccc3C=C2CCC1 ; *c1ccc(*)cc1 ; */C=C/c1ccc(*)cc1 ; */C=C/C=C/c1ccc(*)cc1]

In each of these π-conjugated systems, the aromatic ring may have a substituent Z. The substituent is preferably an electron-withdrawing group. Examples of the electron-withdrawing group include fluorine, chlorine, iodine, a trifluoromethyl group, an acetyl group, and a nitro group. The moiety having the electron-withdrawing group is not limited, but is preferably in the ortho position of the O-Su group. Preferred conjugated systems include

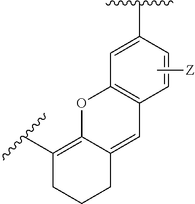

wherein Z is an electron-withdrawing group. More preferred conjugated systems include

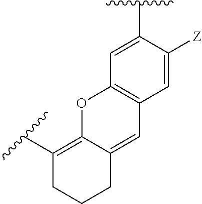

wherein Z is an electron-withdrawing group. The electron-withdrawing group is as described above.

COOH_OH produced by the enzymatic response of CHO_βgal exhibits strong emission under basic conditions, but the emission intensity is reduced in neutral or weakly acidic solutions commonly found in an in-vivo environment. Thus, the concentration (20 μM) of CHO_βgal used for treatment needs to be higher than the concentration (1 to 5 μM) of a common molecular probe. Meanwhile, when an electron-withdrawing group is introduced into the backbone of CHO_βgal, the emission intensity is also increased in neutral or weakly acidic solutions, and the resulting compound functions as a probe which, at the same concentration as a common molecular probe, also exhibits strong emission in neutral or weakly acidic solutions.

The compound according to the second embodiment of the present invention is preferably a compound represented by any one of the following chemical formulas:

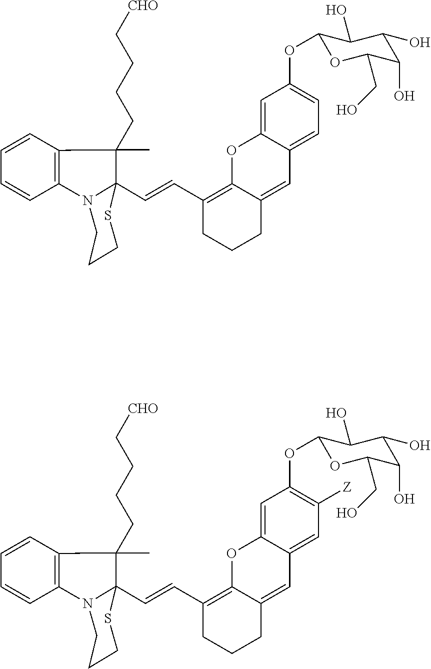

wherein Z is an electron-withdrawing group.

A compound according to a third embodiment of the present invention is characterized by having a formyl group, a quenching moiety, and a luminescent moiety that is non-luminous due to the quenching moiety, wherein the luminescent moiety may be rendered luminous by converting the formyl group to a carboxy group by an aldehyde dehydrogenase to allow the quenching moiety to dissociate.

The luminescence mechanism of the compound of the present invention is described based on CHO_OH, which is an example of a compound of the present invention in which the quenching moiety is S, and the luminescent moiety is a structure including indole and a xanthene skeleton linked via a vinylene group. The formyl group can be converted to a carboxy group by the aldehyde dehydrogenase ALDH1A1, a biomarker that is highly expressed in cells with stemness properties. The conversion to a carboxy group is accompanied by dissociation of the quenching moiety S (dissociation of the bond between S and C) to form an iminium, which is then conjugated with the xanthene skeleton via the vinylene to change the luminescent moiety from non-luminous to luminous. This compound emits fluorescence when irradiated with near-infrared light. According to such a mechanism, stem cells containing ALDH1A1 can be detected.

The aldehyde dehydrogenase, the formyl group, the quenching moiety, and the luminescent moiety are as described above.

A compound according to a fourth embodiment of the present invention is characterized by being represented by the following formula:

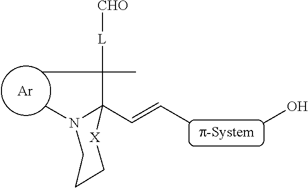

wherein Ar is an aromatic ring, X is an oxygen atom, a nitrogen atom, a sulfur atom, or a selenium atom, L is a linker, and the π-system is a π-conjugated system.

The aromatic ring Ar, the linker L, and the π-conjugated system are as described above.

The luminescence mechanism of the compound according to the fourth embodiment of the present invention is described based on CHO_OH, which is an example of a compound of the present invention in which the quenching moiety is S and the luminescent moiety is a structure including indole and a xanthene skeleton linked via a vinylene group. The formyl group can be converted to a carboxy group by the aldehyde dehydrogenase ALDH1A1, a biomarker that is highly expressed in cells with stemness properties. The conversion to a carboxy group is accompanied by dissociation of the quenching moiety S (dissociation of the bond between S and C) to form an iminium, which is then conjugated with the xanthene skeleton via the vinylene to change the luminescent moiety from non-luminous to luminous. This compound emits fluorescence when irradiated with near-infrared light. According to such a mechanism, stem cells containing ALDH1A1 can be detected.

The luminescence mechanism of the compound according to the second embodiment of the present invention is shown by the following scheme. The substrate moiety in CHO_Su can be detached by a substrate-degrading enzyme in cancer stem cells to produce CHO_OH. The formyl group can be converted to a carboxy group by an aldehyde dehydrogenase that is a biomarker highly expressed in cells with stemness properties. The conversion to a carboxy group is accompanied by dissociation of the quenching moiety S (dissociation of the bond between S and C) to form an iminium, which is then conjugated with the I-conjugated system via the vinylene to change the luminescent moiety from non-luminous to luminous. This compound emits fluorescence when irradiated with near-infrared light. According to such a mechanism, only cancer stem cells, which correspond to both cancer cells containing the substrate-degrading enzyme and stem cells containing the aldehyde-degrading enzyme, can be detected.

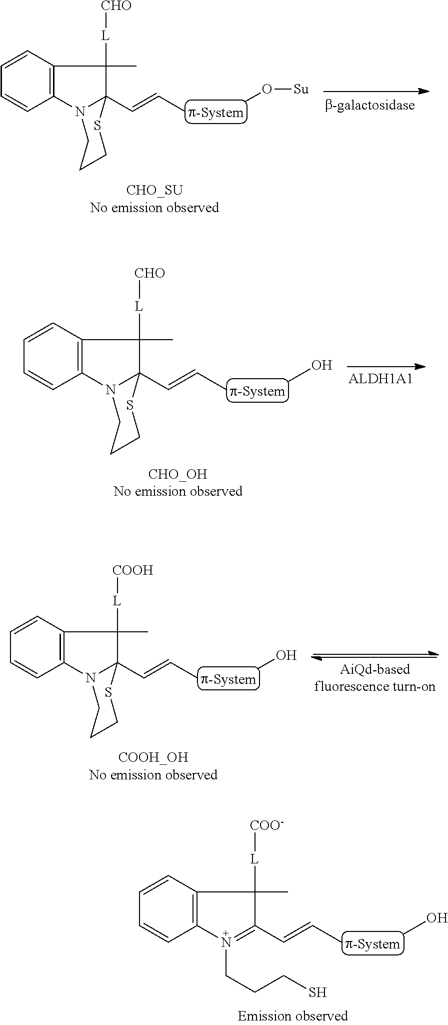

In each of the compounds according to the first to fourth embodiments of the present invention, the indole ring structure on the left side can be synthesized by a known method (M. Oe, et al. ACS Sens. 2021, 6, 3320. DOI: 10.1021/acssensors.1c01136).

The cancer stem cell detection reagent of the present invention is characterized by containing the compound according to the first or second embodiment of the present invention. The cancer stem cell detection reagent may contain a solvent, a buffer, a pH adjuster, a stabilizer, a serum, an antibacterial agent, a nutrient, or other components that are commonly contained in detection reagents. Examples of the solvents include water, dimethylsulfoxide (DMSO), and dimethylformamide (DMF). In the cancer stem cell detection reagent, the concentration of the compound of the present invention is preferably 0.1 to 50 μM, more preferably 10 to 20 μM.

The cancer stem cell detection reagent of the present invention may be a component of a cancer stem cell detection kit. The cancer stem cell detection kit may contain a buffer, a test tube, a petri dish, a positive control sample, a negative control sample, or other components, in addition to the cancer stem cell detection reagent of the present invention.

The method for detecting cancer stem cells of the present invention is characterized by including administering the cancer stem cell detection reagent of the present invention to cells; and irradiating the cells with near-infrared light to detect cancer stem cells by emission from the compound.

The reagent may be administered to any cells that may be human cells or non-human animal cells. Examples of the non-human animals include primates, rodents (e.g., mice, rats), and rabbits. The tissue from which the cells are derived is not limited, with examples including prostate, uterus, ovary, lung, skin, stomach, intestine, liver, pancreas, gallbladder, blood cells, and brain. The administration target cells may be cultured cells or cells contained in tissue. Examples of the tissue containing cells include cultured tissue, biological tissue obtained from test subjects, and tissue sections.

The cancer stem cell detection reagent can be administered to cells by any method, including in vitro administration and in vivo administration to cells in test subjects. In vitro administration may be carried out, for example, by immersing cells or tissue containing cells in a medium or buffer containing the cancer stem cell detection reagent, or applying or dropping the cancer stem cell detection reagent onto tissue containing cells. In vivo administration may be carried out by application, injection, oral administration, rectal administration, vaginal administration, or inhalation.

The method for detecting cancer stem cells is not limited, but after the cancer stem cell detection reagent is administered to cells, the cells are preferably left to stand for 5 to 180 minutes, more preferably 30 to 60 minutes, and then irradiated with near-infrared light. The wavelength of the near-infrared light is preferably 640 to 700 nm. By observing the emission upon the irradiation with near-infrared light, it is possible to determine that cancer stem cells are present at the site where the emission is observed. The emission wavelength is preferably 670 to 800 nm.

When the cancer stem cell detection reagent is administered to cells in vitro, in vitro detection can be performed by irradiating cells in a container such as a petri dish with near-infrared light and observing the emission. When the cancer stem cell detection reagent is administered to cells in vivo, in vivo detection can be performed by irradiating the area in the test subject where cells are present with near-infrared light and observing the emission. For in vivo detection, cells inside the body can be observed non-invasively by adjusting the wavelength of the near-infrared light to be applied and the detection wavelength.

The compound of the present invention can be used for screening cancer therapeutic drugs. The method for screening cancer therapeutic drugs may include:
(i) administering the compound of the present invention to cancer stem cells in the presence or absence of a test substance;
(ii) irradiating the cancer stem cells with near-infrared light to detect emission from the compound; and
(iii) selecting the test substance as a cancer therapeutic drug when the emission intensity of the compound decreases in the presence of the test substance compared to its absence.

The test substance used in step (i) may be any candidate substance for a cancer therapeutic drug. The cancer stem cells may be any cells that have been confirmed to be cancer stem cells, and may be derived from any animal or organ. Steps (i) and (ii) can be carried out under the same conditions as those for the in vitro administration and in vitro detection methods described for the method for detecting cancer stem cells.

In step (iii), the emission intensity of the compound in the presence and absence of the test substance is compared. A decrease in emission intensity in the presence of the test substance compared to its absence means that the test substance inactivated or eliminated the cancer stem cells, and therefore the test substance can be selected as a cancer therapeutic drug. To select the test substance as a cancer therapeutic drug, the decrease in emission intensity of the compound in the presence of the test substance compared to its absence is preferably 50% or more, more preferably 80% or more. High-throughput screening is possible by performing steps (i) to (iii) for tens to thousands of test substances simultaneously.

The stem cell detection reagent of the present invention and the method for detecting stem cells of the present invention are characterized by including the compound according to the third or fourth embodiment of the present invention. For the details of the stem cell detection reagent and the method for detecting stem cells, those described for the cancer stem cell detection reagent of the present invention and the method for detecting cancer stem cells of the present invention are directly applicable.

### EXAMPLES

A compound (CHO_βgal) of the present invention was synthesized according to the following scheme.

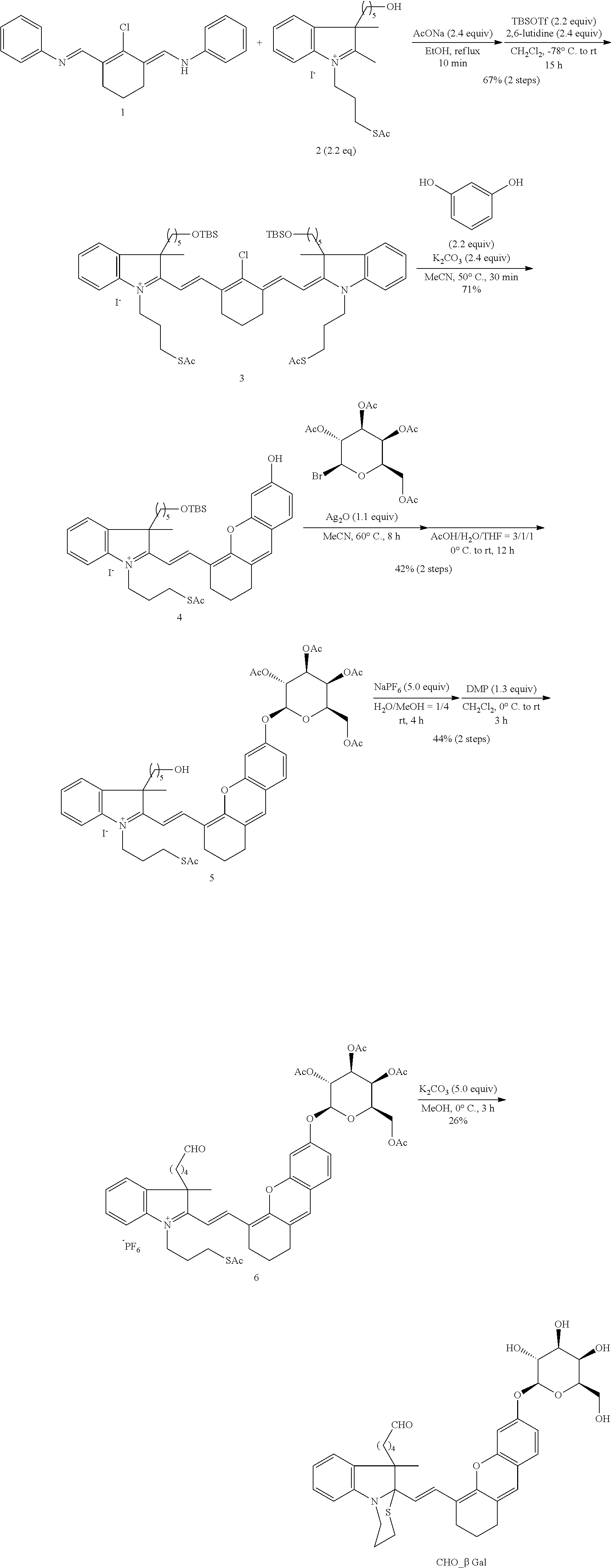

All solvents and reagents were special-grade or first-grade reagents from Nacalai Tesque, Inc., Tokyo Chemical Industry Co., Ltd., FUJIFILM Wako Pure Chemical Corporation, Kishida Chemical Co., Ltd., or Aldrich and were used as received, except for those specifically described below.

A pH 2.0 buffer solution was prepared by dissolving hydrochloric acid and potassium chloride in Milli-Q. A pH 2.8-5.2 buffer solution was prepared by dissolving citric acid and sodium dihydrogen phosphate in Milli-Q. A pH 6.0-8.6 buffer solution was prepared by dissolving sodium dihydrogen phosphate and disodium hydrogen phosphate in Milli-Q. A pH 9.2-10.6 buffer solution was prepared by dissolving sodium bicarbonate and sodium carbonate in Milli-Q.

### Synthesis Example 1 (Synthesis of Cyanine Dye 3)

A Schlenk flask heat-dried under reduced pressure was charged with Compound 2 (1.6 g, 3.4 mmol), N-[[2-chloro-3-[(phenylamino)methylene]-1-cyclohexen-1-yl]methylene]benzenamine 1 (0.50 g, 1.6 mmol), sodium acetate (0.31 mg, 3.7 mmol), and EtOH (9 mL) in a nitrogen atmosphere, and the mixture was heated under reflux for 10 minutes. The organic solvent was removed under reduced pressure, and the resulting product was dissolved in CH₂Cl₂ (15 mL), to which 2,6-lutidine (0.40 g, 3.7 mmol) and tert-butyldimethylsilyl trifluoromethanesulfonate (TBSOTf, 0.90 g, 3.4 mmol) were then added at −78° C., followed by stirring at room temperature for 15 hours. The organic solvent was removed under reduced pressure, and the residue was purified by alumina column chromatography (Al₂O₃ (neutral), CH₂Cl₂/MeOH=100:0 to 50:1) to obtain Cyanine Dye 3 as a green solid (1.2 g, 1.0 mmol, 67%).

### Identification of Cyanine Dye 3:

mp 109-112° C.

IR (ATR) 3046, 2926, 2854, 2695, 1743, 1693, 1563, 1486, 1437, 1381, 1320, 1257, 1228, 1177, 1123, 1094, 1016, 922, 853, 798, 774, 741, 715, 610, 523, 511 cm⁻¹.

¹HNMR (400 MHz, CDCl₃, 25° C.) δ−0.03 (s, 12H), 0.54-0.61 (m, 2H), 0.78-0.80 (m, 2H), 0.83 (s, 9H), 0.83 (s, 9H), 1.17-1.24 (m, 4H), 1.31-1.37 (m, 4H), 1.68 (s, 3H), 1.69 (s, 3H), 2.01 (t, J=6.0 Hz, 2H), 2.08-2.14 (m, 6H), 2.25-2.33 (m, 2H), 2.37 (s, 6H), 2.74 (t, J=6.4 Hz, 4H), 3.03 (t, J=6.9 Hz, 4H), 3.43-3.48 (m, 4H), 4.17-4.23 (m, 4H), 6.29 (d, J=14.2 Hz, 2H), 7.19 (d, J=8.2 Hz, 2H), 7.24-7.26 (m, 2H), 7.33 (d, J=6.9 Hz, 2H), 7.41 (t, J=8.2 Hz, 2H), 8.33 (d, J=13.7 Hz, 2H).

¹³CNMR (100 MHz, CDCl₃, 25° C.) δ−5.3, 18.3, 20.7, 24.1, 25.7, 26.0, 26.2, 26.4, 27.6, 28.2, 28.3, 30.7, 32.1, 32.1, 42.0, 42.1, 43.3, 53.9, 62.6, 62.7, 101.9, 110.6, 119.4, 122.2, 122.5, 125.4, 128.0, 129.0, 139.4, 142.8, 143.9, 150.6, 150.6, 171.0, 195.5.

HRMS (ESI) calcd for C₆₀H₉₂ClN₂O₄S₂Si₂ ([M]⁺) 1059.5720, found 1059.5703.

### Synthesis Example 2 (Synthesis of Hemicyanine Dye 4)

A Schlenk flask heat-dried under reduced pressure was charged with resorcinol (0.24 g, 2.2 mmol), potassium carbonate (0.33 g, 2.4 mmol), and CH₃CN (10 mL) in a nitrogen atmosphere, and the mixture was stirred at room temperature for 10 minutes. A solution of Cyanine Dye 3 (1.2 g, 1.0 mmol) in CH₃CN (10 mL) was added to the resulting mixture, followed by stirring at 50° C. for 30 minutes. The organic solvent was removed under reduced pressure, and the residue was purified by silica gel column chromatography (CH₂Cl₂/MeOH=50:1 to 20:1) to obtain Hemicyanine Dye 4 as a blue solid (0.29 g, 0.37 mmol, 71%).

### Identification of Hemicyanine Dye 4:

mp 103-108° C.

IR (ATR) 3457, 3016, 2970, 2944, 2851, 1738, 1436, 1366, 1228, 1217, 1113, 1054, 915, 834, 753, 636, 542, 521, 511 cm⁻¹.

¹HNMR (400 MHz, CDCl₃, 25° C.) δ−0.08 (s, 3H), −0.08 (s, 3H), 0.50-0.57 (m, 1H), 0.78 (s, 9H), 0.81-0.86 (m, 1H), 1.16-1.21 (m, 2H), 1.31 (t, J=6.9 Hz, 2H), 1.76 (s, 3H), 1.95 (t, J=9.2 Hz, 2H), 2.10-2.14 (m, 2H), 2.26 (t, J=8.2 Hz, 2H), 2.39 (s, 3H), 2.67-2.71 (m, 2H), 2.77 (t, J=6.0 Hz, 2H), 3.04 (t, J=6.9 Hz, 2H), 3.41 (t, J=6.0 Hz, 2H), 4.12-4.19 (m, 2H), 6.11 (d, J=14.2 Hz, 1H), 7.15 (d, J=7.8 Hz, 1H), 7.22 (d, J=8.7 Hz, 1H), 7.28-7.32 (m, 3H), 7.38-7.43 (m, 3H), 8.55 (d, J=14.2 Hz, 1H).

¹³CNMR (100 MHz, CDCl₃, 25° C.) δ−5.4, 18.2, 20.5, 20.6, 24.1, 24.2, 25.5, 25.9, 25.9, 26.2, 27.5, 28.4, 28.7, 30.7, 32.2, 42.5, 43.2, 54.4, 62.6, 99.9, 100.0, 103.0, 107.2, 110.3, 114.9, 118.7, 122.6, 125.8, 125.8, 128.8, 128.9, 138.6, 139.5, 142.4, 142.5, 155.7, 163.6, 163.6, 195.3.

HRMS (ESI) calcd for C₄₀H₅₄NO₄SSi ([M]⁺) 672.3537, found 672.3538.

### Synthesis Example 3 (Synthesis of Hemicyanine Dye 5)

A Schlenk flask heat-dried under reduced pressure was charged with Hemicyanine Dye 4 (0.42 g, 0.30 mmol), 2,3,4,6-tetraacetyl-β-D-galactopyranosyl bromide (0.14 g, 0.33 mmol), silver oxide (76 mg, 0.33 mmol), and MeCN (40 mL) in a nitrogen atmosphere, and the mixture was stirred at 60° C. for 8 hours. The organic solvent was removed under reduced pressure, and the residue was dissolved in AcOEt (20 mL), followed by washing the organic layer with a saturated sodium bicarbonate aqueous solution (20 mL) and a saturated sodium chloride aqueous solution (20 mL). The organic layer was dried over magnesium sulfate, and the solvent was removed under reduced pressure. The resulting product was dissolved at 0° C. in an AcOH/H₂O/THE solution (42 mL, v:v:v=3:1:1), followed by stirring at room temperature for 12 hours. The organic solvent was removed under reduced pressure, CH₂Cl₂ (20 mL) was added to the residue, the organic layer was washed with a saturated sodium bicarbonate aqueous solution (20 mL) and a saturated sodium chloride aqueous solution (20 mL), and the organic layer was dried over magnesium sulfate. The solvent was removed under reduced pressure, and the residue was purified by silica gel column chromatography (CH₂Cl₂/MeOH=20:1 to 10:1) to obtain Hemicyanine Dye 5 as a blue solid (0.13 g, 0.13 mmol).

### Identification of Hemicyanine Dye 5:

mp 120-124° C.

IR (ATR) 3123, 2985, 2961, 2694, 1951, 1690, 1557, 1486, 1394, 1322, 1305, 1255, 1123, 1177, 1053, 1046, 973, 922, 853, 792, 746, 636, 605, 536, 511 cm⁻¹.

¹HNMR (400 MHz, CDCl₃, 25° C.) δ 0.54-0.66 (m, 1H), 0.75-0.86 (m, 1H), 1.20-1.29 (m, 2H), 1.35-1.40 (m, 2H), 1.80 (s, 3H), 1.93-1.98 (m, 2H), 2.03 (s, 3H), 2.07 (s, 3H), 2.10 (s, 3H), 2.16 (t, J=7.8 Hz, 2H), 2.20 (s, 3H), 2.30-2.31 (m, 1H), 2.38 (s, 3H), 2.36-2.39 (m, 1H), 2.79-2.83 (m, 4H), 3.05 (t, J=6.9 Hz, 2H), 3.43-3.49 (m, 2H), 4.09-4.16 (m, 2H), 4.27-4.32 (m, 2H), 4.44-4.48 (m, 1H), 5.22-5.24 (m, 1H), 5.37 (d, J=7.8 Hz, 1H), 5.50-5.55 (m, 2H), 6.58 (d, J=5.0 Hz, 0.5H), 6.62 (d, J=5.5 Hz, 0.5H), 6.95-7.01 (m, 2H), 7.19 (s, 1H), 7.37-7.44 (m, 3H), 7.48-7.52 (m, 2H), 8.68 (d, J=14.6 Hz, 1H).

¹³CNMR (100 MHz, CDCl₃, 25° C.) δ 20.2, 20.5, 20.6, 20.7, 20.8, 24.0, 25.5, 25.6, 26.1, 28.2, 28.2, 29.3, 30.6, 31.7, 42.3, 55.5, 60.9, 61.9, 62.0, 66.7, 68.5, 68.6, 70.6, 70.9, 98.4, 104.6, 115.6, 117.5, 119.2, 122.5, 125.7, 126.0, 127.5, 128.7, 128.8, 129.4, 132.8, 140.4, 142.0, 153.9, 159.2, 161.2, 169.9, 170.1, 170.3, 170.4, 177.0, 196.6.

HRMS (ESI) calcd for C₄₈H₅₈NO₁₃S ([M]⁺) 888.3623, found 888.3621.

### Synthesis Example 4 (Synthesis of Hemicyanine Dye 6)

A round-bottom flask was charged with Hemicyanine Dye (0.13 g, 0.13 mmol), MeOH (6.0 mL), Milli-Q water (1.5 mL), and NaPF₆ (0.11 g, 0.65 mmol), and the mixture was stirred at room temperature for 4 hours. CH₂Cl₂ (20 mL) was added to the reaction solution, and the organic layer was washed with Milli-Q water (20 mL×2). The organic layer was dried over sodium sulfate, and the solvent was removed under reduced pressure to obtain 0.13 g of a blue solid. The obtained solid was dissolved in CH₂Cl₂ (5.5 mL), and 0.3 M Dess-Martin periodinane (0.34 mL, 0.10 mmol) was added at 0° C., followed by stirring at room temperature for 3 hours. CH₂Cl₂ (20 mL) was added to the reaction solution, and the organic layer was washed with a mixed solution of a saturated sodium bicarbonate aqueous solution (10 mL) and a saturated sodium thiosulfate aqueous solution (10 mL), and then again with a saturated sodium bicarbonate aqueous solution (10 mL×3). The organic layer was dried over magnesium sulfate, and the solvent was removed under reduced pressure. The residue was purified by silica gel column chromatography (CH₂Cl₂:MeOH=20:1) and the organic solvent was removed under reduced pressure to obtain Hemicyanine Dye 6 as a blue solid (35 mg, 34 μmol, 44%).

### Identification of Hemicyanine Dye 6:

mp 135-138° C.

IR (ATR) 3744, 2983, 2961, 1952, 1691, 1599, 1555, 1486, 1395, 1322, 1306, 1254, 1213, 1177, 1158, 1123, 1107, 1046, 971, 928, 909, 890, 852, 744, 610, 558, 532, 523, 510 cm⁻¹.

¹HNMR (400 MHz, CDCl₃, 25° C.) δ 0.55-0.64 (m, 1H), 0.87-0.96 (m, 1H), 1.47-1.50 (m, 2H), 1.80 (s, 1.5H), 1.81 (s, 1.5H), 1.93-1.96 (m, 2H), 2.03 (s, 3H), 2.07 (s, 3H), 2.11 (s, 1.5H), 2.11 (s, 1.5H), 2.14-2.18 (m, 2H), 2.21 (s, 3H), 2.29-2.31 (m, 2H), 2.33 (s, 1.5H), 2.35 (s, 1.5H), 2.37-2.39 (m, 2H), 2.73-2.76 (m, 4H), 3.00 (t, J=6.9 Hz, 2H), 4.10-4.11 (m, 1H), 4.28-4.39 (m, 4H), 5.22-5.27 (m, 1H), 5.33 (d, J=8.2 Hz, 0.5H), 5.40 (d, J=7.8 Hz, 0.5H), 5.51-5.55 (m, 2H), 6.51 (d, J=14.6 Hz, 1H), 6.93-6.96 (m, 1H), 7.03 (s, 1H), 7.18 (s, 1H), 7.35-7.37 (m, 2H), 7.42-7.44 (m, 1H), 7.47-7.52 (m, 2H), 8.67 (d, J=15.1 Hz, 1H), 9.60 (t, J=1.3 Hz, 0.4H), 9.64 (t, J=1.3 Hz, 0.6H).

¹³CNMR (100 MHz, CDCl₃, 25° C.) δ 20.2, 20.6, 20.7, 20.7, 20.8, 21.5, 21.6, 23.8, 23.9, 26.1, 28.1, 30.7, 43.2, 44.3, 44.4, 55.4, 60.4, 60.9, 66.7, 68.0, 68.5, 68.5, 70.7, 70.8, 98.4, 98.6, 104.8, 115.7, 115.7, 117.4, 122.5, 127.6, 128.6, 129.4, 132.8, 142.0, 145.8, 154.0, 158.8, 159.4, 169.7, 169.9, 170.1, 170.3, 170.4, 176.7, 195.5, 202.1, 202.5.

HRMS (ESI) calcd for C₄₈H₅₆NO₁₃S ([M]⁺) 886.3467, found 886.3454.

### Example 1 (Synthesis of CHO_βgal)

Hemicyanine Dye 6 (35 mg, 34 μmol) was dissolved in MeOH (4.0 mL), K₂CO₃ (23 mg, 0.17 mmol) was added at 0° C. to the solution, followed by stirring for 3 hours. CH₂Cl₂ (20 mL) was added to the reaction solution, and the organic layer was washed with a saturated sodium bicarbonate aqueous solution (10 mL×2). The organic layer was dried over sodium sulfate, and the solvent was removed under reduced pressure to obtain CHO_βgal as a deep green solid (6.0 mg, 8.9 μmol).

### Identification of CHO_βgal:

mp 137-140° C.

IR (ATR) 3456, 3016, 2970, 2921, 2850, 2135, 1738, 1572, 1435, 1365, 1258, 1228, 1217, 1206, 1091, 1070, 896, 798, 741, 584, 576, 558, 542, 526, 512 cm⁻¹.

¹HNMR (400 MHz, CDCl₃, 25° C.) δ 0.76-0.87 (m, 2H), 1.53-1.61 (m, 2H), 1.70 (s, 3H), 1.90-1.97 (m, 2H), 2.10-2.24 (m, 6H), 2.39-2.43 (m, 4H), 3.45-3.48 (m, 2H), 3.57-3.60 (m, 5H), 3.81-3.87 (m, 2H), 5.26-5.29 (m, 2H), 6.05-6.06 (m, 1H), 6.49-6.54 (m, 3H), 6.69-6.75 (m, 3H), 6.91-6.94 (m, 2H), 7.07-7.09 (m, 1H), 9.55-9.56 (m, 1H).

HRMS (ESI) calcd for C₃₈H₄₆NO₈S ([M+H]⁺) 676.2939, found 676.2951.

A comparative compound (CHO—OH) was synthesized according to the following scheme.

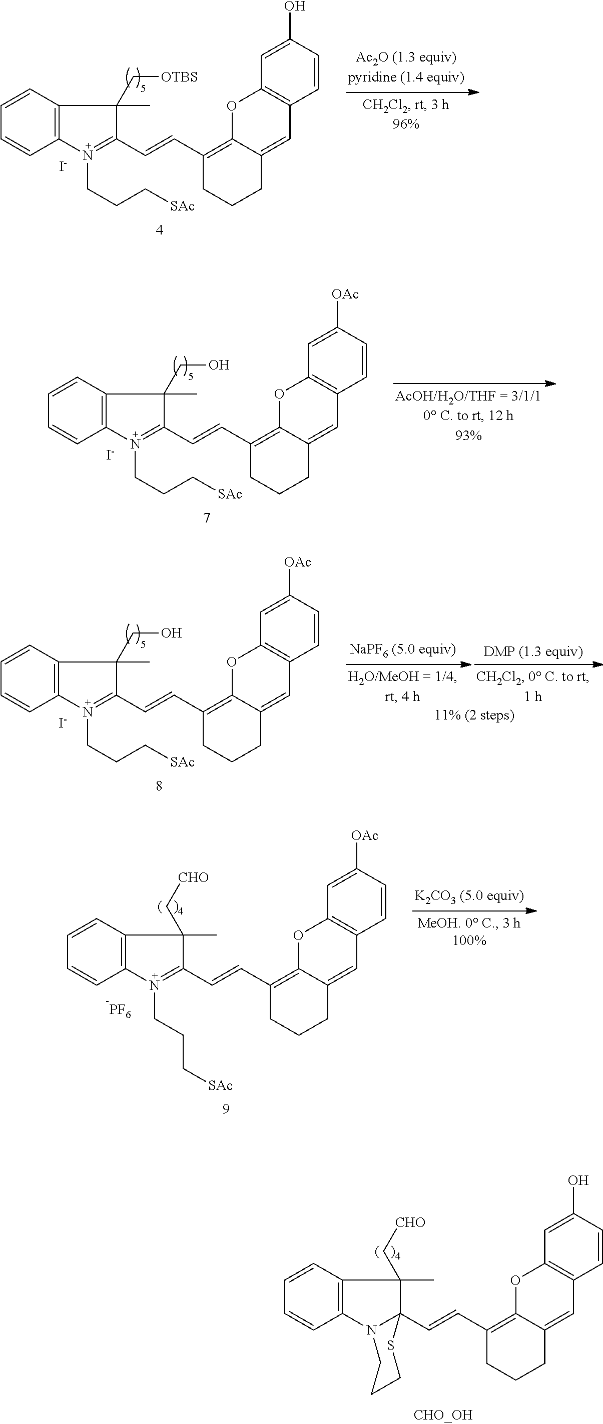

### Synthesis Example 5 (Synthesis of Hemicyanine Dye 7)

A 25-mL round-bottom flask was charged with Hemicyanine Dye 4 (24 mg, 30 μmol) and CH₂Cl₂ (5 mL) to prepare a solution. Then, acetic anhydride (3.6 μL, 39 μmol) and pyridine (3.3 μL, 42 μmol) were added to the solution, followed by stirring at room temperature for 3 hours. The organic layer was washed with water (10 mL) and a saturated sodium chloride aqueous solution (10 mL), the organic layer was dried over magnesium sulfate, and the solvent was removed under reduced pressure to obtain Hemicyanine Dye 7 as a blue solid (24 mg, 29 mmol).

### Identification of Hemicyanine Dye 7:

Mp 92-95° C.

IR (ATR) 2981, 2959, 2924, 2852, 2666, 2054, 1951, 1691, 1556, 1485, 1394, 1322, 1255, 1177, 1122, 1053, 973, 922, 853, 675, 635, 610, 524, 518, 510 cm⁻¹.

¹HNMR (400 MHz, CDCl₃, 25° C.) δ−0.07 (s, 6H), 0.50-0.56 (m, 1H), 0.79 (s, 9H), 0.79-0.84 (m, 1H), 1.18-1.23 (m, 2H), 1.30-1.35 (m, 2H), 1.78 (s, 3H), 1.97 (t, J=6.4 Hz, 2H), 2.16 (t, J=7.3 Hz, 2H), 2.29-2.31 (m, 2H), 2.33 (s, 3H), 2.38 (s, 3H), 2.75 (t, J=6.0 Hz, 2H), 2.82-2.83 (m, 2H), 3.09 (t, J=6.9 Hz, 2H), 3.43 (t, J=6.4 Hz, 2H), 4.54-4.62 (m, 2H), 6.79 (d, J=15.1 Hz, 2H), 7.01-7.04 (m, 1H), 7.08-7.20 (m, 2H), 7.39 (d, J=8.2 Hz, 1H), 7.44 (d, J=4.1 Hz, 1H), 7.48-7.54 (m, 2H), 8.64 (d, J=15.1 Hz, 1H).

¹³CNMR (100 MHz, CDCl₃, 25° C.) δ−5.4, 18.3, 20.1, 21.2, 24.0, 24.1, 25.7, 25.9, 26.0, 26.1, 28.2, 28.5, 29.5, 30.7, 31.0, 32.0, 42.6, 44.9, 55.7, 62.5, 106.7, 109.4, 113.1, 116.0, 119.1, 119.7, 122.3, 127.9, 128.1, 129.6, 130.5, 131.1, 140.5, 142.0, 146.0, 152.6, 160.1, 168.9, 177.6, 195.7, 207.1.

HRMS (ESI) calcd for C₄₂H₅₆NO₅SSi ([M]⁺) 714.3643, found 714.3635.

### Synthesis Example 6 (Synthesis of Hemicyanine Dye 8)

A 200-mL round-bottom flask was charged with Hemicyanine Dye 7 (0.11 g, 0.13 mmol), which was then dissolved at 0° C. in an AcOH/H₂O/THE solution (15 mL, v:v:v=3:1:1), followed by stirring at room temperature for 12 hours. The organic solvent was removed under reduced pressure, CH₂Cl₂ (20 mL) was added to the residue, and the organic layer was washed with a saturated sodium bicarbonate aqueous solution (20 mL) and a saturated sodium chloride aqueous solution (20 mL). The organic layer was dried over magnesium sulfate, and the solvent was removed under reduced pressure to obtain Hemicyanine Dye 8 as a blue solid (85 mg, 0.12 mmol).

### Identification of Hemicyanine Dye 8:

mp 108-111° C.

IR (ATR) 2932, 1767, 1690, 1532, 1449, 1371, 1256, 1152, 1121, 1057, 1029, 952, 802, 754, 637, 623, 597, 572, 562, 547, 527, 512, 503 cm⁻¹.

¹HNMR (400 MHz, CDCl₃, 25° C.) δ 0.57-0.63 (m, 1H), 0.87-0.95 (m, 1H), 1.19-1.29 (m, 2H), 1.35-1.42 (m, 2H), 1.78 (s, 3H), 1.94-2.00 (m, 2H), 2.14-2.19 (m, 2H), 2.32-2.37 (m, 2H), 2.34 (s, 3H), 2.38 (s, 3H), 2.75 (t, J=5.2 Hz, 2H), 2.79-2.85 (m, 2H), 3.07 (t, J=7.3 Hz, 2H), 3.47 (t, J=6.0 Hz, 2H), 4.55 (t, J=7.8 Hz, 2H), 6.75 (d, J=15.1 Hz, 1H), 7.00-7.03 (m, 1H), 7.11-7.13 (m, 2H), 7.38 (d, J=8.2 Hz, 1H), 7.44-7.47 (m, 3H), 7.51-7.53 (m, 1H), 8.66 (d, J=15.1 Hz, 1H).

¹³CNMR (100 MHz, CDCl₃, 25° C.) δ 20.1, 21.2, 23.9, 24.1, 25.5, 26.1, 28.3, 28.4, 29.5, 30.7, 31.7, 42.4, 44.8, 55.7, 62.1, 106.6, 109.6, 113.0, 116.0, 119.0, 119.7, 122.4, 127.9, 128.1, 129.5, 130.4, 131.1, 140.6, 141.9, 146.1, 152.6, 153.0, 160.2, 169.1, 177.7, 195.8.

HRMS (ESI) calcd for C₃₆H₄₂NO₅S ([M]⁺) 600.2778, found 600.2788.

### Synthesis Example 7 (Synthesis of Hemicyanine Dye 9)

Hemicyanine Dye 9 was synthesized by the same procedure as in the synthesis of Hemicyanine Dye 6, except that Hemicyanine Dye 8 was used instead of Hemicyanine Dye 5.

### Identification of Hemicyanine Dye 9:

a blue solid (54%):

mp 112-115° C.

IR (ATR) 2979, 1938, 1689, 1531, 1503, 1487, 1451, 1425, 1398, 1286, 1258, 1222, 1209, 1176, 1154, 1122, 1051, 1017, 953, 917, 875, 837, 761, 621, 597, 557, 536, 518, 511 cm⁻¹.

¹HNMR (400 MHz, CDCl₃, 25° C.) δ 0.52-0.63 (m, 1H), 0.85-0.96 (m, 1H), 1.47 (dt, J=15.6, 8.7 Hz, 2H), 1.79 (s, 3H), 1.93-1.99 (m, 2H), 2.12-2.16 (m, 2H), 2.29-2.38 (m, 4H), 2.34 (s, 3H), 2.38 (s, 3H), 2.74-2.77 (m, 4H), 3.01 (t, J=7.3 Hz, 2H), 4.39 (t, J=7.3 Hz, 2H), 6.54 (d, J=14.6 Hz, 1H), 7.02-7.05 (m, 1H), 7.16-7.20 (m, 2H), 7.39-7.53 (m, 5H), 8.66 (d, J=14.6 Hz, 1H), 9.60-9.61 (m, 1H).

¹³CNMR (100 MHz, CDCl₃, 25° C.) δ 20.1, 21.1, 21.6, 23.7, 23.8, 26.0, 28.1, 28.2, 29.3, 30.7, 42.2, 43.1, 44.4, 55.5, 105.5, 109.8, 112.7, 115.7, 119.4, 119.6, 122.5, 127.9, 128.2, 129.5, 130.1, 132.0, 140.3, 141.8, 146.0, 153.0, 153.1, 160.7, 169.0, 177.2, 195.5, 202.2.

HRMS (ESI) calcd for C₃₆H₄₀NO₅SS ([M]⁺) 598.2622, found 598.2617.

### Comparative Example 1 (Synthesis of Hemicyanine Dye CHO_OH)

CHO_OH was synthesized by the same procedure as in the synthesis of CHO_βgal, except that Hemicyanine Dye 9 was used instead of Hemicyanine Dye 6.

### Identification of CHO_OH:

a greenish blue solid (100%):

mp 135-140° C.

IR (ATR) 3461, 3016, 2970, 2945, 1738, 1574, 1436, 1365, 1290, 1228, 1217, 1206, 1123, 1101, 1018, 911, 840, 741, 542, 525, 511 cm⁻¹.

¹HNMR (400 MHz, CDCl₃, 25° C.) δ 0.62-0.69 (m, 1H), 0.98-1.03 (m, 1H), 1.23-1.28 (m, 2H), 1.45-1.52 (m, 2H), 1.64 (s, 3H), 1.80-1.91 (m, 2H), 1.99-2.04 (m, 1H), 2.15-2.18 (m, 1H), 2.23-2.28 (m, 1H), 2.45-2.49 (m, 1H), 2.57-2.66 (m, 4H), 2.72-2.76 (m, 1H), 2.72-2.76 (m, 1H), 3.72-3.73 (m, 1H), 3.88 (t, J=5.5 Hz, 2H), 5.69 (d, J=13.3 Hz, 1H), 6.49-6.56 (m, 3H), 6.73-6.79 (m, 3H), 7.01-7.04 (m, 1H), 7.15-7.22 (m, 1H), 7.91 (d, J=13.3 Hz, 1H), 9.56 (t, J=5.5 Hz, 0.7H), 9.63 (t, J=3.2 Hz, 0.3H).

¹³CNMR (100 MHz, CDCl₃, 25° C.) δ 21.4, 21.9, 24.2, 24.6, 25.3, 26.5, 27.8, 28.7, 30.0, 34.8, 40.8, 41.4, 43.2, 51.4, 51.5, 94.4, 97.2, 103.6, 107.3, 115.7, 116.9, 122.0, 127.4, 128.2, 128.6, 128.6, 129.9, 130.1, 137.4, 140.4, 144.1, 159.3, 202.3, 205.6.

HRMS (ESI) calcd for C₃₂H₃₆NO₃S ([M+H]⁺) 514.2410, found 514.2412.

A compound (CHO_OH—F) was synthesized according to the following scheme.

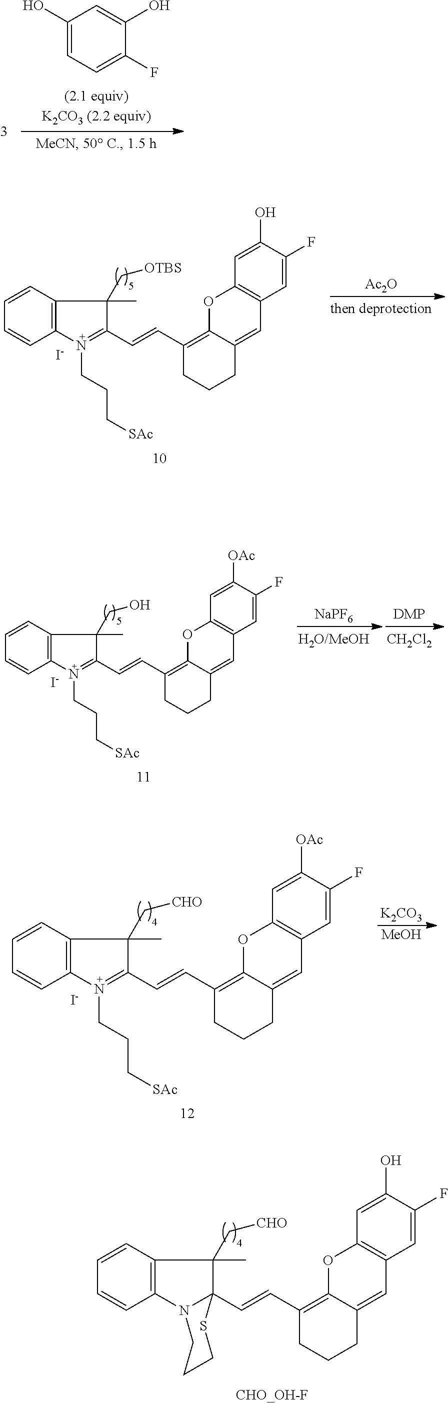

### Synthesis Example 9 (Synthesis of Hemicyanine Dye 10)

A Schlenk flask heat-dried under reduced pressure was charged with 4-fluororesorcinol (21 mg, 0.16 mmol), potassium carbonate (24 mg, 0.17 mmol), and MeCN (3 mL) in a nitrogen atmosphere, and the mixture was stirred at room temperature for 10 minutes. A solution of Cyanine Dye 3 (92 mg, 77 μmol) in MeCN (5 mL) was added to the reaction solution, followed by stirring at 50° C. for 1.5 hours. The organic solvent was removed under reduced pressure, and the residue was purified by silica gel column chromatography (CHCl₃/MeOH=10:1) to obtain Compound 10 as a blue solid (11 mg, 13 μmol, 18%).

### Identification of Hemicyanine Dye 10:

¹H NMR (400 MHz, CDCl₃, 25° C.) δ−0.07 (s, 3H), −0.07 (s, 3H), 0.54-0.67 (m, 1H), 0.79 (s, 9H), 0.82-0.89 (m, 1H), 1.12-1.21 (m, 2H), 1.28-1.35 (m, 2H), 1.63 (s, 3H), 1.87-1.95 (m, 2H), 1.97-2.05 (m, 2H), 2.16-2.24 (m, 2H), 2.38 (s, 3H), 2.64 (t, J=5.5 Hz, 2H), 2.70 (t, J=5.5 Hz, 2H), 2.96 (t, J=7.1 Hz, 2H), 3.42 (y, J=6.4 Hz, 2H), 3.82 (t, J=7.3 Hz, 2H), 5.65 (d, J=12.8 Hz), 6.69 (d, J=7.3 Hz, 1H), 6.79 (d, J=7.8 Hz, 1H), 6.96 (d, J=10.1 Hz, 1H), 7.04 (t, J=6.9 Hz, 1H), 7.23-7.26 (m, 2H), 7.37 (s, 1H), 8.00 (d, J=13.8 Hz, 1H).

HRMS (ESI) calcd for C₄₀H₅₃FNO₄SSi ([M]⁺) 690.3443, found 690.3441.

### Synthesis Example 10 (Synthesis of Hemicyanine Dye 11)

After Compound 10 (37 mg, 45 μmol) was dissolved in CH₂Cl₂ (5 mL), acetic anhydride (4.7 μL, 50 μmol) and pyridine (4.4 μL, 55 μmol) were added, and the mixture was stirred at room temperature for 4 hours. The organic layer was washed with water (10 mL) and a saturated sodium chloride aqueous solution (10 mL), the organic layer was dried over magnesium sulfate, and the solvent was removed under reduced pressure to obtain an acetylated compound (7.7 mg, 9.0 μmol, 20%).

The obtained acetylated compound was dissolved in a mixed solution of AcOH, H₂O, and THE (5 mL, v:v:v=3:1:1) cooled to 0° C., and the solution was then stirred at room temperature for 3 hours. The organic solvent was removed under reduced pressure, the residue was dissolved in CH₂Cl₂ (20 mL), and the organic layer was washed with a saturated sodium bicarbonate aqueous solution (20 mL) and a saturated sodium chloride aqueous solution (20 mL). The organic layer was dried over magnesium sulfate, and the solvent was removed under reduced pressure to obtain Compound 11 as a blue solid (6.6 mg, 8.9 μmol, 97%).

### Identification of Hemicyanine Dye 11:

HRMS (ESI) calcd for C₃₆H₄₁FNO₃S ([M]⁺) 618.2684, found 618.2676.

### Synthesis Example 11 (Synthesis of Hemicyanine Dye 12)

Compound 11 (6.6 mg, 8.9 μmol), MeOH (4 mL), Milli-Q water (1 mL), and NaPF₆ (15 mg, 89 μmol) were added and stirred overnight at room temperature. CH₂Cl₂ (20 mL) was added to the reaction solution, and the organic layer was washed with Milli-Q water (20 mL×2). The organic layer was dried over magnesium sulfate, and the solvent was removed under reduced pressure to obtain a blue solid (6.3 mg).

The obtained solid was dissolved in CH₂Cl₂ (2 mL), and Dess-Martin periodinane (4.3 mL, 10 μmol) was added at 0° C., followed by stirring at room temperature for one hour. CH₂Cl₂ (20 mL) was added to the reaction solution, and the organic layer was washed with a mixed solution of a saturated sodium bicarbonate aqueous solution (10 mL) and a saturated sodium thiosulfate aqueous solution (10 mL), and then again with a saturated sodium bicarbonate aqueous solution (10 mL×3). The organic layer was dried over magnesium sulfate, and the solvent was removed under reduced pressure. The residue was purified by silica gel column chromatography (CHCl₃:MeOH=10:1), and the organic solvent was removed under reduced pressure to obtain Compound 12 as a blue solid (4.0 mg, 5.2 μmol, 60%).

### Identification of Hemicyanine Dye 12:

¹H NMR (400 MHz, CDCl₃, 25° C.) δ 0.55-0.62 (m, 2H), 0.91-0.97 (m, 1H), 1.42-1.49 (m, 2H), 1.78 (s, 3H), 1.94-2.04 (m, 2H), 2.13-2.21 (m, 2H), 2.32 (s, 3H), 2.28-2.35 (m, 4H), 2.41 (s, 3H), 2.72-2.74 (m, 2H), 2.93 (t, J=6.6 Hz, 2H), 3.16 (t, J=6.4 Hz, 2H), 4.30 (t, J=6.9 Hz, 2H), 7.12-7.15 (m, 2H), 7.45 (d, J=2.8 Hz), 7.52-7.56 (m, 3H), 7.68-7.72 (m, 2H), 8.60 (d, J=14.7 Hz, 1H).

HRMS (ESI) calcd for C₃₆H₃₉FNO₅S ([M]⁺) 616.2527, found 616.2534.

### Synthesis Example 12 (Synthesis of CHO_OH—F)

Compound 12 (4.0 mg, 5.2 μmol) was dissolved in MeOH (3 mL), and K₂CO₃ (2.3 mg, 16 μmol) was added, followed by stirring for 3 hours. CH₂Cl₂ (20 mL) was added to the reaction solution, and the organic layer was washed with water (10 mL×2). The organic layer was dried over magnesium sulfate, and the solvent was removed under reduced pressure to obtain CHO_OH—F as a deep green solid (1.1 mg, 2.1 μmol, 39%).

Identification of hemicyanine dye CHO_OH—F:

HRMS (ESI) calcd for C₃₂H₃₅FNO₃S ([M+H]⁺) 532.2316, found 532.2318.

The compounds synthesized in Example 1 and Comparative Example 1 were subjected to the following assessments.

### <Ultraviolet-Visible Absorption Spectrum, Fluorescence Spectrum>

The ultraviolet-visible absorption spectrum was measured with a UH-5300 spectrophotometer (Hitachi, Ltd.). The fluorescence spectrum was measured with RF-6000 (Shimadzu Corporation).

The compound synthesized in Example 1 (CHO_βgal) or Comparative Example 1 (CHO_OH) was weighed with a precision balance and dissolved in DMSO to prepare a dye solution (2.0 mM). Each dye solution (20 μL) was dissolved in a buffer solution (1980 μL) to prepare a sample solution (20 μM), and the absorption and fluorescence spectra of each sample solution were measured. The excitation wavelength used to measure the fluorescence spectra was 660 nm for CHO_βgal and 680 nm for CHO_OH.

### <Enzyme Responsiveness Experiment>

ALDH1A1 was dissolved in a 0.1 M phosphate buffer solution (pH 7.4) to prepare an ALDH1A1 solution. β-Galactosidase was dissolved in a 0.1 M phosphate buffer solution (pH 7.4) to prepare a β-galactosidase solution. A phosphate buffer solution (1580 μL) containing potassium chloride, dithiothreitol (DTT), and nicotinamide adenine dinucleotide (NAD⁺) was mixed with a solution (20 μL, 2.0 mM) of CHO_βgal in DMSO. After this mixed solution was stirred at 37° C. for 10 minutes, the ALDH1A1 solution (200 UL, 2.0 μM) and the β-galactosidase solution (200 μL, 200 U) were added, and the changes in fluorescence intensity over time were measured. When only one of the enzyme solutions was added, 200 μL of a 0.1 M phosphate buffer solution was added simultaneously with the enzyme solution (200 μL). The final concentrations of CHO_βgal, potassium chloride, DTT, NAD⁺, ALDH1A1, and β-galactosidase were 20 μM, 100 mM, 2 mM, 1 mM, 200 nM, and 10 U/mL, respectively. FIG. 1-1 shows the measurement results.

FIG. 1-1 shows changes in fluorescence intensity when CHO_βgal (20 μM, phosphate buffer solution (pH=7.4)) synthesized in Example 1 was treated with (a) no enzyme (control), (b) ALDH1A1 alone, (c) β-galactosidase alone, and (d) ALDH1A1 and β-galactosidase. ALDH1A1 (200 nM), β-galactosidase (20 U). Excitation wavelength: 660 nm, Detection wavelength: 710 nm Time elapsed: 0 min (red), 10 min (orange), 20 min (yellow), 30 min (yellowish green), 45 min (green), 60 min (light blue), 90 min (blue), 120 min (dark blue), 180 min (purple). (e) Changes over time in fluorescence intensity normalized to emission intensity at 0 min (FL/FL_{0 min}) No enzyme (gray), ALDH1A1 alone (light green), β-galactosidase alone (orange), ALDH1A1 and β-galactosidase (red).

When CHO_βgal is treated with either ALDH1A1 or β-galactosidase, little emission occurs, although it is about four times the emission of the original dye (FIG. 1-1(b), (c), and (e)). In contrast, when CHO_βgal is treated with both ALDH1A1 and β-galactosidase, the emission intensity increases 39-fold compared to before the treatment (FIG. 1-1(d)). These results demonstrate that CHO_βgal exhibits strong emission in the presence of both ALDH1A1 and β-galactosidase.

ALDH1A1 was dissolved in a 0.1 M phosphate buffer solution (pH 7.6) to prepare an ALDH1A1 solution. A phosphate buffer solution (1780 μL) containing potassium chloride, dithiothreitol (DTT), and nicotinamide adenine dinucleotide (NAD⁺) was mixed with a DMSO solution (20 μL, 0.50 mM) of CHO_OH—F synthesized in Synthesis Example 12. After this mixed solution was stirred at 37° C. for 15 minutes, the ALDH1A1 solution (200 μL, 0.50 μM) was added, and the changes in fluorescence intensity over time were measured. The final concentrations of CHO_OH—F, potassium chloride, DTT, NAD⁺, and ALDH1A1 were 5 μM, 100 mM, 2 mM, 1 mM, and 50 mM, respectively. To perform an inhibition test, disulfiram (inhibitor of ALDH1A1) was dissolved in DMSO, and the solution was diluted with a phosphate buffer solution to prepare a disulfiram solution (10 mM). The ALDH1A1 solution (100 μL, 1.0 μM) and the disulfiram solution (100 μL, 10 mM) were mixed and stirred at 37° C. for 15 minutes to inhibit the ALDH1A1 activity. A phosphate buffer solution (1780 μL) containing potassium chloride, dithiothreitol (DTT), and nicotinamide adenine dinucleotide (NAD⁺) was mixed with the DMSO solution (20 μL, 0.50 mM) of CHO_OH—F. The solution was stirred at 37° C. for 15 minutes, and the ALDH1A1-containing solution (200 μL) in which the ALDH1A1 activity had been inhibited was added to the resulting solution to measure changes in fluorescence intensity over time. The final concentration of disulfiram was 500 μM.

When CHO_OH—F was treated with ALDH1A1, the relative emission intensity after 150 minutes was 2.86, whereas it decreased to 1.89 when the inhibitor was added, and was 1.01 when the enzyme was not added. This demonstrates that CHO_OH—F has ALDH1A1 responsiveness.

FIG. 1-2 shows changes in fluorescence intensity of CHO_OH—F synthesized in Synthesis Example 12 and COOH_OH produced by the enzymatic response of CHO_βgal. (b) COOH_OH produced by the enzymatic response of CHO_βgal did not have sufficient fluorescence intensity in the acidic to neutral range, whereas CHO_OH—F synthesized in Synthesis Example 12, in which fluorine had been introduced into the aromatic ring, had high fluorescence intensity. When a substrate moiety is introduced into CHO_OH—F synthesized in Synthesis Example 12, in which fluorine has been introduced into the aromatic ring, the sensitivity can be expected to increase, facilitating the detection of cancer stem cells.

### <Enzyme Selectivity Experiment>

A solution (20 μL, 2.0 mM) of a dye in DMSO and a 0.1 M phosphate buffer solution (pH 7.4, 1780 μL) were mixed and stirred at 37° C. for 10 minutes. A solution (200 μL) containing GSH, BSA, an amino acid (Cys, Glu, Ser, Lys, or Gly), or a reactive oxygen species (O₂⁻, H₂O₂, OH, or ClO⁻) was added to this mixed solution, and the changes in fluorescence intensity over time were measured. The solutions containing the reactive oxygen species were prepared according to the previous reports ((a) D. Yang, H.-L. Wang, Z.-N. Sun, N.-W. Chung, J.-G. Shen, J. Am. Chem. Soc. 2006, 128, 6004-6005. (b) X. Xie, X. Yang, T. Wu, Y. Li, M. Li, Q. Tan, X. Wang, B. Tang, Anal. Chem. 2016, 88, 8019-8025.). The final concentrations of GSH, BSA, amino acids, and reactive oxygen species were 1 mM, 1 μM, 100 μM, and 100 μM, respectively, and the final concentration of CHO_βgal was 20 μM. FIG. 2 shows the measurement results.

FIG. 2 shows the responsiveness (37° C., 60 min) of CHO_βgal synthesized in Example 1 to biochemical substances and oxidizing agents. The results are normalized to the emission intensity immediately after the addition of each reactant. The concentrations of CHO_βg, al, GSH, BSA, amino acids, reactive oxygen species, ALDH1A1, and β-galactosidase: 20 μM, 1 mM, 1 μM, 100 μM, 100 μM, 200 nM, and 10 U/mL, respectively, Excitation wavelength: 660 nm, Detection wavelength: 710 nm.

To demonstrate that CHO_βgal is stable and not responsive to amino acids or reactive chemicals, particularly oxidizing agents, commonly present in the body, CHO_βgal was treated with amino acids having mercapto, amino, and carboxy groups, as well as oxidizing agents such as hydrogen peroxide (FIG. 2). When CHO_βgal was treated with BSA, the emission intensity slightly increased, but this increase was slight compared to the increase in emission intensity when it was treated with ALDH1A1 alone or with ALDH1A1 and β-galactosidase.

### <Cell Culture>

The SUIT-2 cells, KATO-3 cells, A549 cells, H1299 cells, OVK-18 cells, NIH3T3 cells, and HUVEC cells used were purchased from the JCRB Cell Bank of the National Institutes of Biomedical Innovation, Health and Nutrition.

SUIT-2 cells, OVK-18 cells, and NIH3T3 cells were cultured in DMEM medium (Dulbecco's modified Eagle's medium) containing 10% fetal bovine serum and 1% penicillin-streptomycin in air containing 5% CO₂ at 37° C. KATO-3 cells were cultured in RPMI-1640 medium (Roswell Park Memorial Institute-1640) containing 10% fetal bovine serum and 1% penicillin-streptomycin. HUVEC cells were cultured in endothelial cell basal medium containing Supplement Mix (PromoCell, Heidelberg, Germany).

### <Cytotoxicity Investigation>

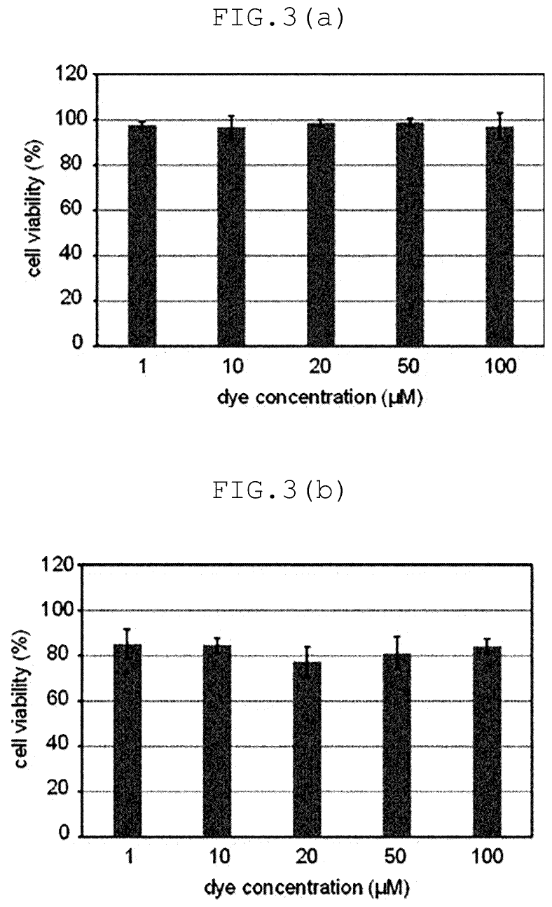

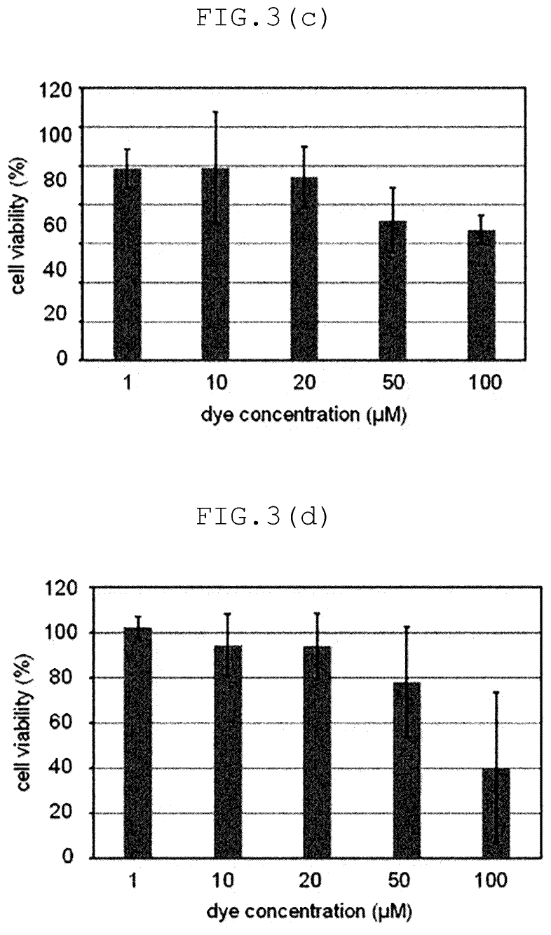

SUIT-2 cells, KATO-3 cells, NIH3T3 cells, and HUVEC cells were seeded at 3.0×10⁴, 8.0×10⁴, 5.0×10⁴, and 5.0×10⁴ cells/well, respectively, in a 96-well plate, which was sprayed with a solution (100 μL) of CHO_βgal diluted to a concentration of 1, 10, 20, 50, or 100 μM with DMEM (for SUIT-2 cells, NIH3T3 cells), RPMI-1640 (for KATO-3 cells), or endothelial cell basal medium (for HUVEC cells). After culturing at 37° C. for 24 hours, the cells were washed once with a phosphate buffer solution, and the medium (100 μL) was added. Then, 100 μL of a MTT solution (0.5 mg/mL) diluted in the medium was added. After this mixture was cultured at 37° C. for 4 hours, the medium was removed and the cells were washed with a phosphate buffer solution. The precipitated purple solid was dissolved in DMSO (200 μL) and the absorbance at 535 nm was measured using a microplate reader 800TS (BioTek). As KATO-3 cells had a weak adhesive force to the bottom of the plate, the washing with the phosphate buffer solution after the incubation with the MTT solution was omitted. FIG. 3 shows the measurement results.

FIG. 3 shows the results of a cytotoxicity test of CHO_βgal to (a) SUIT-2, (b) KATO-III, (c) HUVEC, and (d) NIH-3T3.

To assess the cytotoxicity of CHO_βgal to the cells, an MTT assay was performed (FIG. 3). At concentrations equal to or lower than 20 μM, the cell viability observed was approximately 80% or higher even after 24 hours. Since all the cell experiments in the present example were performed with a 20 μM CHO_βgal solution, it is considered that CHO_βgal hardly damaged the cells in the experiments.

<Observation with Confocal Microscope>

Fluorescence imaging was performed using a confocal microscope LSM710 (Carl Zeiss AG). The excitation light used was at 488 nm (detection wavelength: 500-600 nm) and 633 nm (detection wavelength: 647-759 nm).

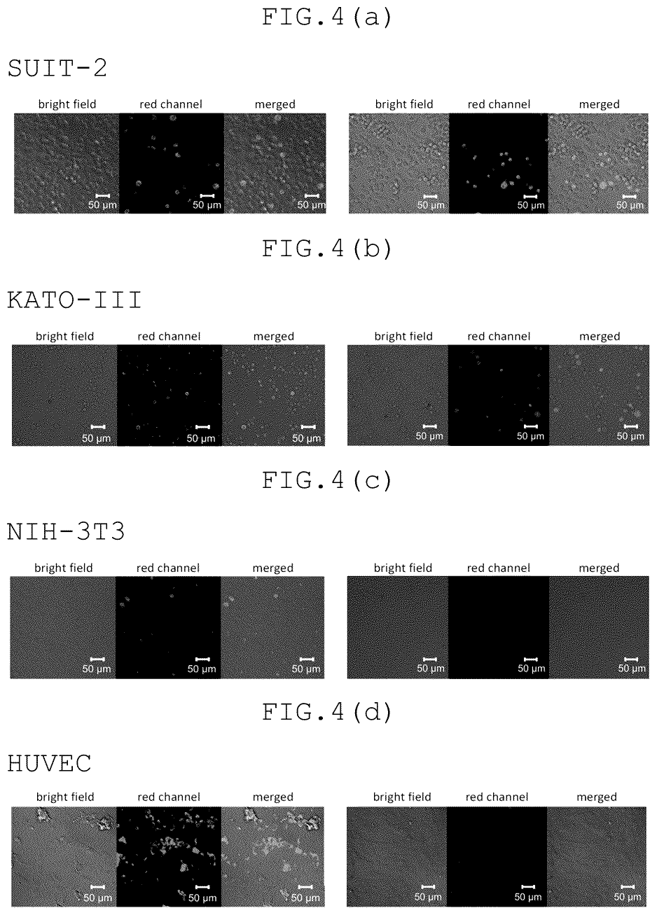

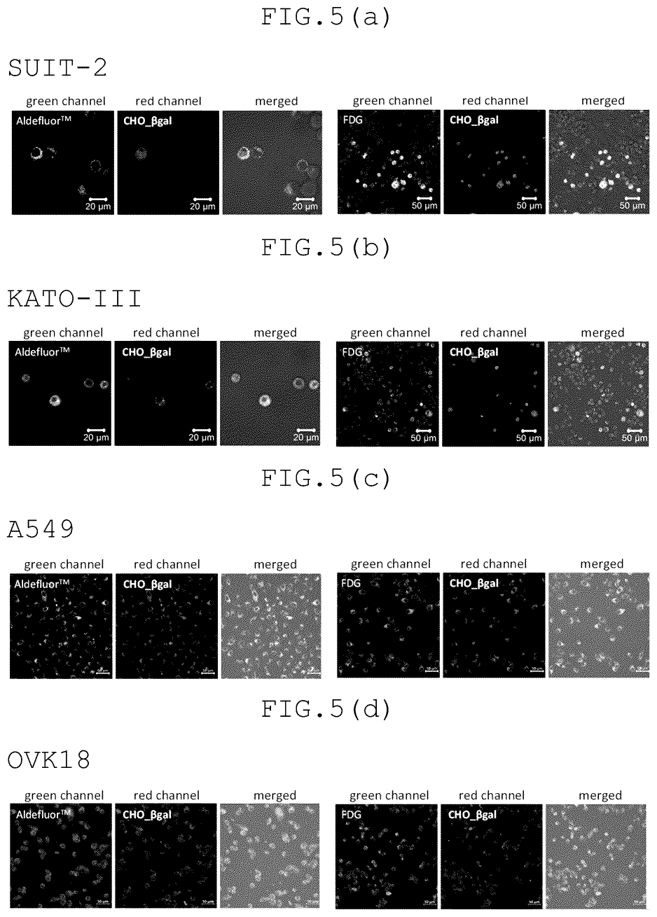

The cells were seeded in a 35 mm micro-Dish (μ-Dish 35 mm, high, ibidi, Germany) and cultured for 24 hours, followed by washing with DMEM (for SUIT-2 cells, NIH3T3 cells, HUVEC cells) or RPMI-1640 (for KATO-3 cells) containing no phenol red and no FBS. A dye and Aldefluor™ (StemCell Technologies, Inc., Canada) or FDG (Sigma-Aldrich, USA) were dissolved in DMSO and diluted in the medium. The cells were treated with 2 mL of the medium containing the dye and Aldefluor™ or FDG and cultured in air containing 5% CO₂ at 37° C. for one hour, followed by observation with the confocal microscope. The number of cells seeded was 10×10⁵ for KATO-3 cells, and 8×10⁵ for the other cell types. The final concentrations of CHO_βgal, Aldefluor™, and FDG were 20 μM, 1 μM, and 20 μM, respectively. The DMSO content was 2%. FIG. 4 and FIG. 5 show the measurement results.

FIG. 4 shows confocal laser microscope images of (a) SUIT-2, (b) KATO-III, (c) NIH-3T3, and (d) HUVEC, each treated with C5S-A (left) and CHO_βgal (right). The excitation light is at 633 nm (detection wavelength: 647-759 nm).

When human pancreatic cancer cells (SUIT-2), human gastric signet ring cell carcinoma-derived cells (KATO-III), and NIH-3T3 and HUVEC cells having ALDH1 activity were treated with C5S-A having ALDH1A1 responsiveness (synthesized as described in M. Oe, et al. ACS Sens. 2021, 6, 3320. DOI: 10.1021/acssensors.1c01136), a certain number of ALDH1A1-positive cells were detected (FIG. 4). On the other hand, when they were treated with CHO_βgal, a certain number of cancer stem cells positive for both ALDH1A1 and β-galactosidase were detected in SUIT-2 and KATO-III cells, but cells exhibiting emission were not detected in NIH-3T3 and HUVEC cells. These results demonstrate that both NSCs and CSCs can be detected based on ALDH1A1 responsiveness alone, and that CHO_βgal can only visualize CSCs without responding to NSCs.

FIG. 5 shows confocal laser microscope images of (a) SUIT-2, (b) KATO-III, (c) A549, and (d) OVK18, each co-stained with CHO_βgal synthesized in Example 1 and Aldefluor™ (left) or with CHO_βgal and FDG (right). In FIG. 5, the excitation light is at 488 nm (detection wavelength: 500-600 nm for Aldefluor™ and FDG) and 633 nm (detection wavelength: 647-759 nm for CHO_βgal).

Commercially available Aldefluor™ and commercially available β-galactosidase activity detection agent fluorescein di-β-D-galactopyranoside (FDG, Sigma-Aldrich, USA) (G. P. Nolan, S. Fiering, J. F. Nicolas, L. A. Herzenberg, Proc. Natl. Acad. Sci. USA, 1988, 85, 2603-2607.) were used to perform co-staining experiments of SUIT-2 and KATO-III (FIGS. 5(a) and 5(b)). It was demonstrated that in both SUIT-2 and KATO-III cells, the emissions of CHO_βgal and Aldefluor™ colocalized in some cells, although they did not overlap in some areas within the cells. It should be noted that using Aldefluor™, which is not suitable for microscopic observation, cells with high ALDH1 activity could be detected in magnified images but were difficult to distinguish at low magnifications (M. Oe, K. Miki, Y. Ueda, Y. Mori, A. Okamoto, Y. Funakoshi, H. Minami, K. Ohe, ACS Sens. 2021, 6, 3320-3329.). When FDG was used, emission was observed throughout the cells of both cell types. However, when the lower detection limit for emission was set higher, a mixture of strongly and weakly emitting cells was observed in both cell types. The results of co-staining with CHO_βgal showed that the strongly emitting cells corresponded to CHO_βgal-positive cells, suggesting that β-galactosidase might be highly expressed in cancer stem cells of these cell types. It has been known that β-galactosidase is expressed in these cells (H. Kubo, Y. Murayama, S. Ogawa, T. Matsumoto, M. Yubakami, T. Ohashi, T. Kubota, K. Okamoto, M. Kamiya, Y. Urano, E. Otsuji, Sci. Rep. 2021, 11, 10664.), but it has not been pointed out that the expression level is localized to some cells within the cell population. The finding that β-galactosidase is highly expressed in cells with high stemness properties was revealed for the first time by using CHO_βgal.

Next, human alveolar basal epithelial adenocarcinoma cells (A549) and human ovarian endometrioid carcinoma-derived cells (OVK18), which are known to include cells with ALDH1A1 activity, were similarly co-stained using Aldefluor™ and FDG, and CHO_βgal (FIGS. 5(c) and 5(d)). In A549 cells, which are known to exhibit a higher ALDH1A1 expression level than SUIT-2 or KATO-III cells, emission was observed throughout the cells, indicating that there was no bias in enzyme activity. Also in OVK18 cells, which are known to exhibit low ALDH1A1 activity, no bias in ALDH1A1 activity was observed. These results demonstrate that although the ALDH1A1 expression level has been assessed by a molecular biological approach, ALDH1A1 may or may not be suitable as a biomarker for CSCs depending on the cell type.

Thus, the dual responsiveness of CHO_βgal not only clarified the suitability of ALDH1A1 as a biomarker for detecting CSCs in certain cancer cells, but also revealed that β-galactosidase can be used as a biomarker for CSCs. It can be a powerful tool in basic research to elucidate the characteristics of CSCs. In addition, the β-galactosidase-responsive moiety can be constructed by chemical modification of the phenolic hydroxy group, and thus could be easily converted to substrates responsive to other external stimuli. The molecular design of CHO_βgal with such diversity will enhance the possibility of discussing the expression levels of other enzymes and chemicals in cancer stem cells in the future, creating a great ripple effect in the field of cancer stem cell research.

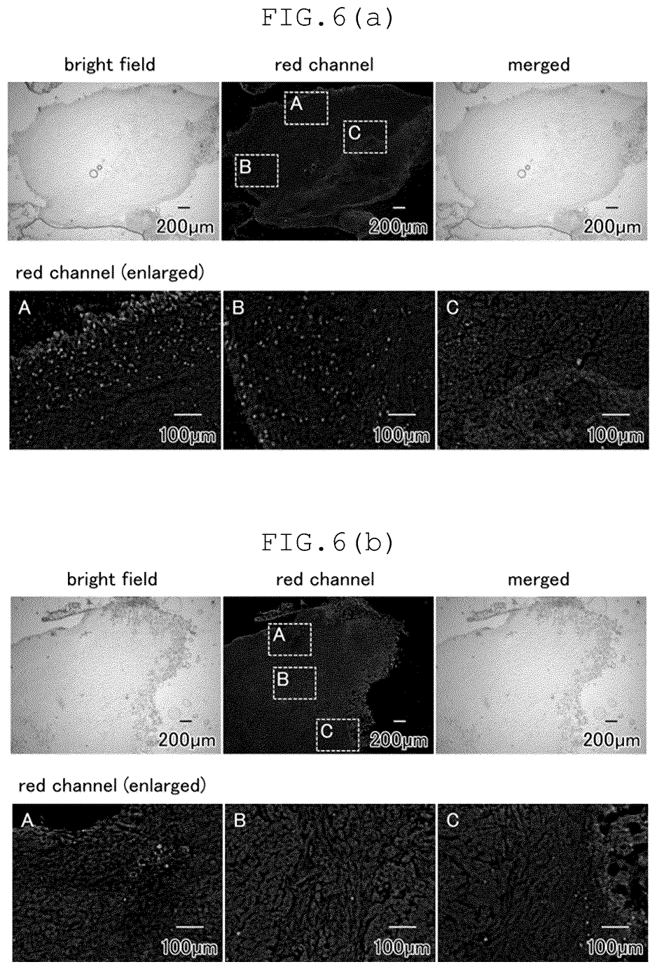

(a) Tumor tissue stained with CHO_βgal and (b) tumor tissue treated with disulfiram, an inhibitor of aldehyde dehydrogenase, and stained with CHO_βgal were observed under a fluorescence microscope. FIG. 6 shows the results. Color development was observed in the absence of the inhibitor of aldehyde dehydrogenase, but no color development was observed in the presence of the inhibitor. It was confirmed that the aldehyde dehydrogenase present in cancer stem cells has a significant impact on the color development.

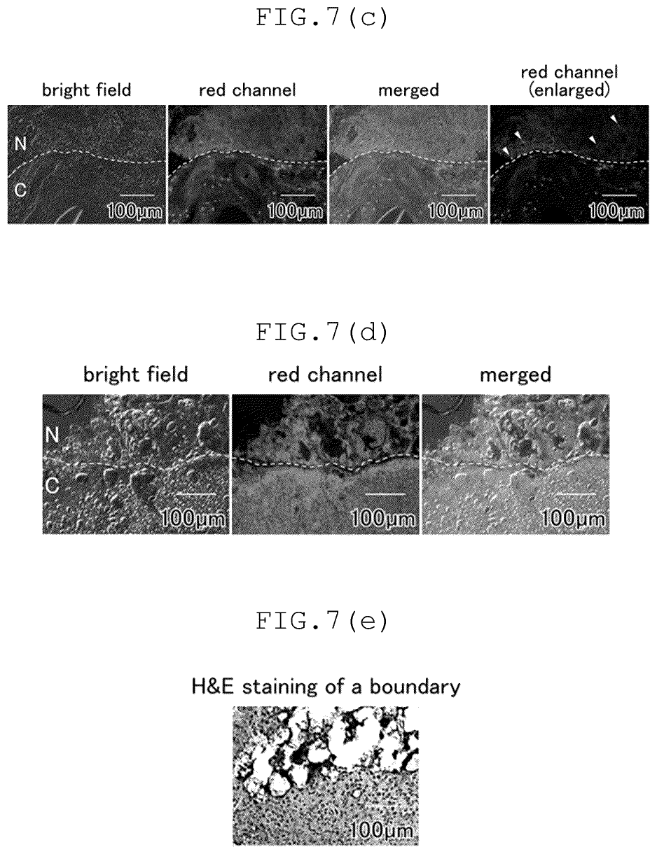

The boundary between tumor tissue and normal cells was stained with (c) CHO_βgal, (d) CSS-A, or (e) hematoxylin-eosin and observed under a fluorescence microscope or optical microscope. FIG. 7 shows the results. The hematoxylin-eosin staining confirmed accurate assessment of the boundary region between cancer tissue and normal tissue. As a result of staining with CSS-A (ALDH1A1-responsive molecular probe), strong fluorescence was observed from both the normal tissue and the cancer tissue, making it difficult not only to distinguish the tissue boundary, but also to identify the bright spots of cancer stem cells. On the other hand, the cancer stem cells were identified by CHO_βgal staining, demonstrating the superiority of CHO_βgal staining. It should be noted that color development was also observed at some points in the normal tissue by CHO_βgal staining, which indicates that the cancer stem cells invaded the normal tissue. Since a certain amount of normal tissue is often removed together during surgery, another advantage of CHO_βgal is that stem cells in normal tissue do not affect the results.

During surgery to remove cancer, a definitive diagnosis of cancer can be made using immunostaining or other similar methods on excised cancer tissue sections. If the sections are simultaneously immersed in a probe solution to assess the abundance of cancer stem cells under a fluorescence microscope, the malignancy (e.g., metastatic potential) of the patient's cancer tissue can be diagnosed. This can provide information that is helpful for the doctor's decision regarding the precision of the surgical procedure and also aids in prognosis prediction. The results of staining tissue sections in FIGS. 6 and 7 demonstrate the following 2 points.
Slight emission was observed from normal tissue, and cancer stem cells within cancer tissue were clearly identified as bright spots.
Cancer stem cells were localized at the periphery of cancer tissue, and some were observed also in normal tissue (cancer stem cell invasion).

### <Lung Metastasis Model>

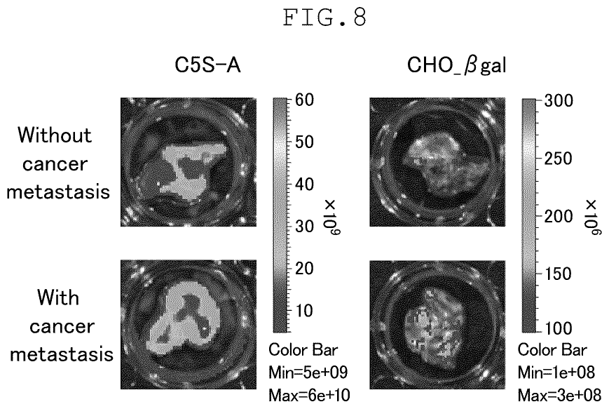

SUIT-2 was administered through the tail vein of mice to prepare lung metastasis models in which SUIT-2 metastasized to the lungs. Since metastasis is thought to develop from cancer stem cells within the cancer cell population, the lung metastasis models can be considered to mainly reflect the colonization of cancer stem cells. When the lungs from mice without SUIT-2 were removed and stained with CSS-A, strong emission was observed (FIG. 8). This is believed to result from responding to pulmonary NSCs as well. Similarly, when the lungs of mice without SUIT-2 were treated with CHO_βgal, only weak emission was observed. On the other hand, when the lungs of mice with SUIT-2 were treated with CSS-A, strong emission was observed as with the unadministered lungs, and thus no clear difference was detected. However, when the lungs of mice with SUIT-2 were treated with CHO_βgal, significant emission was observed, indicating that SUIT-2 colonizing the lungs can be detected. The experiments above show that CHO_βgal can detect cancer stem cells with metastatic potential even in an environment where normal cells coexist.

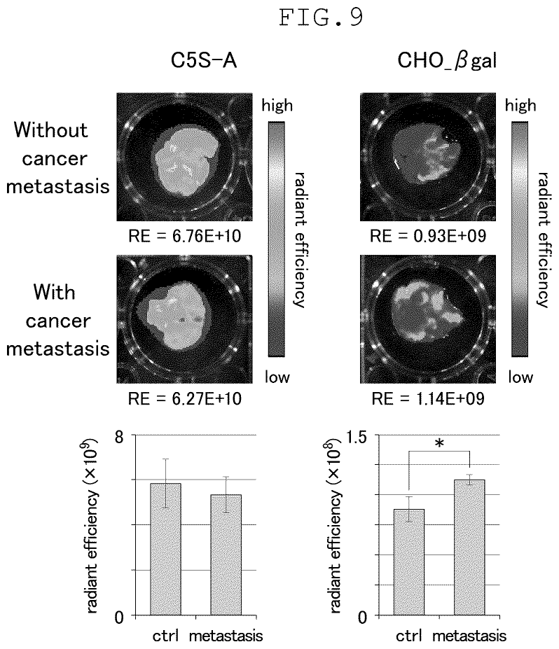

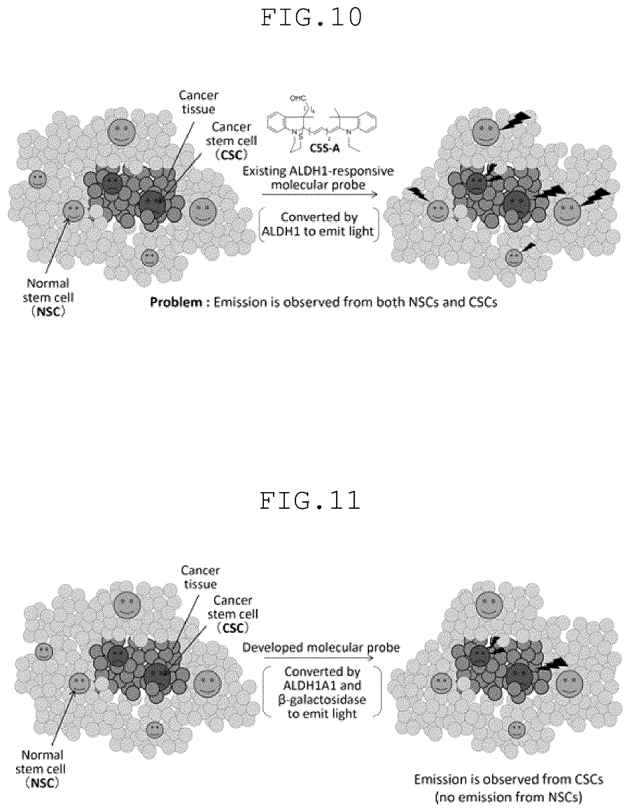

As in the experiments above, separately, SUIT-2 was administered through the tail vein of mice, and the lungs of lung metastasis models in which SUIT-2 metastasized to the lungs (metastasis) and the lungs of mice without SUIT-2 (control) were removed and subjected to CSS-A staining or CHO_βgal staining, followed by measuring the fluorescence imaging data. FIG. 9 shows the results. FIG. 9 also shows bar graphs prepared by performing statistical processing of the imaging data using a T-test and plotting the fluorescence intensity (FL Intensity). In the figure, “RE” represents the fluorescence efficiency. The CSS-A staining showed no significant difference in fluorescence intensity between the two groups, when considering the margin of error, whereas the CHO_βgal staining showed a significant difference with p<0.05 (n=3).

A significant difference between the lungs of the lung metastasis models and the lungs of the control mice was also found in the fluorescence imaging data after CHO_βgal staining in FIG. 8, for which no statistical processing was performed. However, as shown in FIG. 9, the statistical processing revealed a clearer significant difference.

### INDUSTRIAL APPLICABILITY

The compound of the present invention can be used to produce a molecular probe capable of distinguishing NSCs from CSCs, thus making it possible to easily detect cancer stem cells. Therefore, the compound can be expected to have a significant impact on the diagnosis and treatment of cancer in the medical field.

1. A compound, comprising:
a formyl group;
a substrate moiety;
a quenching moiety; and
a luminescent moiety that is non-luminous due to the quenching moiety,
wherein the luminescent moiety may be rendered luminous by detaching the substrate moiety by a substrate-degrading enzyme and also converting the formyl group to a carboxy group by an aldehyde dehydrogenase to allow the quenching moiety to dissociate.

2. The compound according to claim 1,
wherein the substrate moiety is a sugar chain, an amino acid, a peptide chain, a boryl group, a phosphoryl group, an aryl methyl group, or an acyl group.

3. The compound according to claim 2,
wherein the sugar chain is D-galactose, and the sugar chain-degrading enzyme is β-galactosidase.

4. The compound according to claim 1, wherein the luminescent moiety is able to emit near-infrared light.

5. A compound, represented by the following formula: 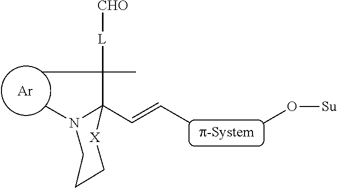
wherein Ar is an aromatic ring, X is an oxygen atom, a nitrogen atom, a sulfur atom, or a selenium atom, L is a linker, Su is a substrate moiety, and the π-system is a π-conjugated system.

6. The compound according to claim 5,
wherein the aromatic ring is represented by the following formula: 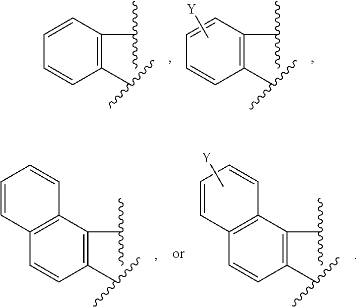
wherein Y is a halogen group, SO₃Na, SO₃H, or COOR.

7. The compound according to claim 5,
wherein the substrate moiety is a sugar chain, an amino acid, a peptide chain, a boryl group, a phosphoryl group, an aryl methyl group, or an acyl group.

8. The compound according to claim 5, wherein the π-conjugated system is represented by the following formula: 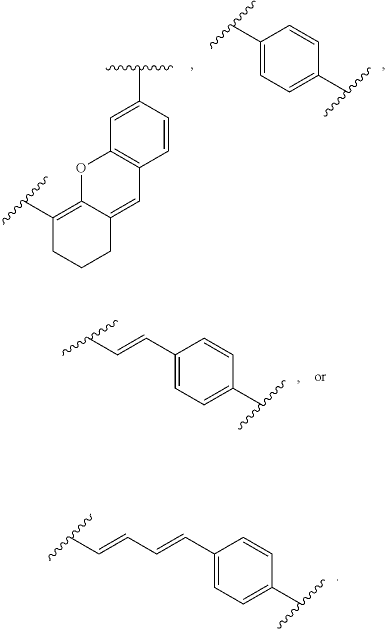 [SMILES: *C1=C2Oc3cc(*)ccc3C=C2CCC1 ; *c1ccc(*)cc1 ; */C=C/c1ccc(*)cc1 ; */C=C/C=C/c1ccc(*)cc1]
wherein the aromatic ring optionally has an electron-withdrawing group.

9. The compound according to claim 5,
wherein the compound is represented by the following formula: 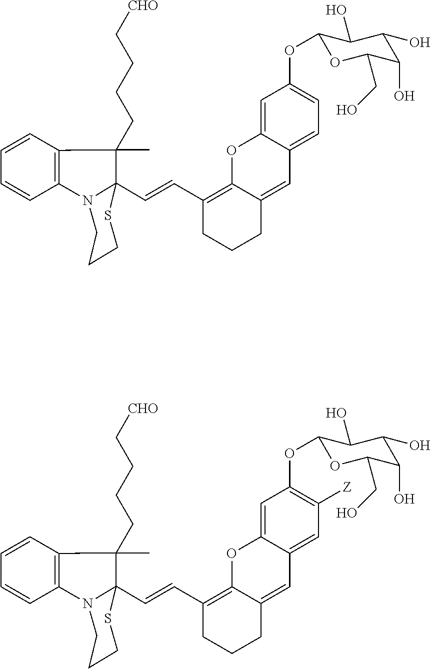
wherein Z is an electron-withdrawing group.

10. A cancer stem cell detection reagent, comprising the compound according to claim 1.

11. A method for detecting cancer stem cells, the method comprising:
administering the cancer stem cell detection reagent according to claim 10 to cells; and
irradiating the cells with near-infrared light to detect cancer stem cells by emission from the compound.

12. The method for detecting cancer stem cells according to claim 11,
wherein the cells are contained in biological tissue and are detected in vivo or in vitro.
